(19) United States
(12) Patent Application Publication
(10) Pub. No.: US 2012/0282618 A1
(43) Pub. Date: Nov. 8, 2012

(54) METHODS AND ASSAYS FOR RISK PREDICTION, DIAGNOSIS, AND ANALYSIS OF MYOCARDIAL INFARCTION, HEART FAILURE AND REDUCED CARDIAC FUNCTION

(75) Inventor: Sakthivel Sadayappan, Naperville, IL (US)

(73) Assignee: LOYOLA UNIVERSITY CHICAGO, Maywood, IL (US)

(21) Appl. No.: 13/464,466

(22) Filed: May 4, 2012

Related U.S. Application Data
(60) Provisional application No. 61/482,280, filed on May 4, 2011.

(51) Int. Cl. G01N 33/566; G01N 21/82; C12Q 1/68

(52) U.S. Cl. 435/6.12; 436/501; 435/7.94

(57) ABSTRACT

Methods and kits adapted for risk prediction, diagnosis, and analysis of myocardial infarction (MI), myocardial failure, and reduced cardiac function such as heart failure. The methods include collecting a sample of human body fluid or tissue from a subject and then identifying the presence of cardiac myosin binding protein-C 25-nucleotide deletion and/or detecting the presence of cardiac myosin binding protein-C, its peptides, its phosphorylation status, and/or its autoantibodies in the human body fluid or tissue.

### CROSS REFERENCE TO RELATED APPLICATIONS

This application claims the benefit of U.S. Provisional Application No. 61/482,280, filed May 4, 2011, the contents of which are incorporated herein by reference.

### BACKGROUND OF THE INVENTION

The present invention generally relates to myocardial infarction (MI). More particularly, this invention relates to methods and systems for prediction of potential near and long-term myocardial failure as well as short and long-term detection of myocardial infarction (MI) and reduced cardiac function.

Coronary heart disease is the most common cause of acute myocardial infarction (AMI), which afflicts about five million people in the U.S. each year. Myocardial infarction (MI) can cause contractile dysfunction, which often persists even after blood flow has been restored. MI is associated with loss of cardiac contractility, altered Ca2+ handling and myofilament protein phosphorylation that leads to increased myofibrillar calcium sensitivity and lower cross-bridge cycling rates. Furthermore, increased intracellular Ca2+ activates protease calpain and leads to proteolytic degradation of contractile proteins. MI can cause either reversible or irreversible damage to the myocardial tissues. Damage initially includes a necrotic core surrounded by a border zone that will either recover or become irreversibly damaged, thus failing to generate tension during systole.

During MI, the sarcomeric proteins are reorganized and degraded, which alters contractile function. These changes correspond to the detection of degraded contractile proteins in the blood. Release of degraded sarcomeric proteins, such as cardiac troponin I (cTnI) and troponin T (cTnT), into the circulatory system occurs during MI, and such sarcomeric proteins are used clinically to determine the severity of myocardial injury. Alterations in the stable states of contractile proteins might underlie cardiac dysfunction after an ischemic period. Although the molecular events that contribute to MI have not been fully elucidated, it is clear that a single signaling pathway does not exclusively regulate MI. Importantly, the degradation of structural proteins resulting in contractile dysfunction as a consequence of MI has not previously been characterized.

MI encompasses all conditions that are caused by a sudden inadequate perfusion of the heart. This can occur through decrease of blood flow or increased demand to the heart. Acute coronary syndrome (ACS) includes electrocardiogram (ECG) ST-segment elevation myocardial infarction (STEMI), non ST-segment elevation myocardial infarction (NSTEMI), and unstable angina. Symptoms can vary from classic crushing chest pain that radiates down the left arm to nondescript jaw or back sensations. Every 25 seconds, approximately one American will experience ACS with an estimated 34% chance of dying within one year subsequent to the ACS event. The ECG provides immediate diagnosis of STEMI, activating a fast 90-minute treatment pathway from first medical contact to opening the blocked coronary artery (i.e., “door to balloon time”). However, ST elevation could be due to other causes such as pericarditis, early repolarization, ventricular hypertrophy, etc. More importantly, life-threatening NSTEMI/UA can still be missed due to a non-diagnostic ECG. Furthermore, the incidence of NSTEMI has increased (126 to 132 per 100,000), while the incidence of STEMI has decreased (121 to 77 per 100,000) from 1997-2005. Moreover, NSTEMI (18.7%-27.6%) shows greater one-year post mortality than STEMI (8.3%-15.4%) [3]. When a patient presents with ACS, but without ST segment elevation, a clinician has to decide on either an early invasive approach or a conservative approach based on assessment of patient's risk. Currently, this decision process can be difficult. The Timing of Intervention in Patients with Acute Coronary Syndromes (TIMACS) trial has shown that early invasive therapy decreases possibility of death/MI/stroke in higher risk NSTEMI/UA patients in comparison to standard care with longer time to invasive therapy. The American Heart Association (AHA) guidelines, the GRACE score, and the Thrombolysis in Myocardial Infarction (TIMI) risk score all use positive biomarkers as indicators of high risk above. Consequently, biomarkers play a crucial role in risk-stratifying a non-STEMI ACS patient for proper care.

The cardiac isoforms of cTnT and cTnI are the gold standard circulatory markers for diagnosis of acute MI. Unlike creatine kinase (CK) and myoglobin, cTnT and cTnI assays are specific to MI. The level of cTnT and cTnI is increased maximally in the blood after MI by the degradation of structural sarcomeric proteins. However, using cTnI as a biomarker has sensitivity and time of release issues. Therefore, an improved method of predicting risk to MI, detecting MI, and determining the severity of the damage to the heart after MI is desirable.

### BRIEF DESCRIPTION OF THE INVENTION

The present invention provides methods and kits for risk prediction, diagnosis, and analysis of myocardial infarction (MI), myocardial failure, and reduced cardiac function such as heart failure.

According to a first aspect of the invention, the method includes collecting a sample of human body fluid or tissue from a subject for genomic DNA extraction and then identifying the presence of cardiac myosin binding protein-C 25-nucleotide deletion.

According to a second aspect of the invention, the method includes collecting a sample of human body fluid or tissue from a subject and then detecting the presence of cardiac myosin binding protein-C, its peptides, its phosphorylation status, and/or its autoantibodies.

According to a third aspect of the invention, the kit includes an immunoassay solution and a means for detecting the presence of cardiac myosin binding protein-C, its peptides, its phosphorylation status, and/or its autoantibodies in human body fluids or tissue.

A technical effect of the invention is the ability to analyze a human heart by detecting a cardiac myosin binding protein-C gene mutation, cardiac myosin binding protein-C, its cleaved peptides including the N′-specific 40 kDa fragments, its phosphorylation status, or its autoantibodies as earlier predictor/indicator of heart failure in subject's body fluids or tissue.

Other aspects and advantages of this invention will be better appreciated from the following detailed description.

### BRIEF DESCRIPTION OF THE DRAWINGS

FIG. 1 represents a cardiac myosin binding protein-C (cMyBP-C) gene as a normal 403 bps band or a mutation-specific 378 bps band with a 25-nucleotide deletion.

FIG. 2A represents differences between cardiac MyBP-C (cMyBP-C) and skeletal MyBP-C (sMyBP-C). Roman numerals indicate the position of polyclonal antibodies that were used in trials described herein against cMyBP-C.

FIGS. 2B-C represent SDS-PAGE and Western blot analyses of wild type rats total heart proteins analyzed with rabbit anti-cMyBP-C₂₋₁₄ antibody to determine the increase in cMyBPC and its fragments over time in an effluent.

FIG. 2D represents Western blot analyses showing the release of other myofilament proteins into the effluent at different time points.

FIGS. 2E-F represent tissue homogenates from used heart tissues at different time points resolved on 10% SDS-PAGE and analyzed by Western blot with rabbit anti-cMyBP-C₂₋₁₄ antibodies. A predominant nonspecific (NS) protein, which was not released into phosphate buffered saline (PBS), was identified.

FIG. 2G represents a 40-kDa N-terminal cMyBP-C fragment as consistent in human (H), rat (R) and mouse (M) heart effluents, but absent in cMyBP-C null (t/t) and systemic knockout (−/−) hearts.

FIG. 2H represents Western blot analyses using Rabbit anti-cMyBP-C₂₋₁₄ antibodies showing increased 40-kDa fragments in H₂O₂-treated neonatal rat ventricular card iomyocytes.

FIGS. 3A-C represent Western blot analyses showing a cMyBP-C degradation profile in vitro using anti-cMyBP-C antibodies that were raised against C5, C8-C9 and C10 domains.

FIG. 4A represents Western blots of effluent samples with Rabbit anti-cMyBP-C₂₋₁₄ antibody.

FIG. 4B represents Western blot analysis of released cMyBP-C and its fragments using phospho-specific antibodies.

FIG. 5A represents Western blot analyses using total heart proteins from human (H), rat (R) and mouse (M) ventricular tissue with rabbit anti-cMyBP-C₂₋₁₄ antibodies.

FIG. 5B represents SDS-PAGE and Western blot analyses carried out with rabbit anti-cMyBP-C₂₋₁₄ antibodies. Results show release of NS protein in the effluent.

FIG. 5C represents silver staining showing the presence of two proteins at a NS 70-kDa region (SilverQuest, Invitrogen).

FIG. 5D represents Western blot analyses using 5 ng of partial purified NS proteins with rabbit anti-cMyBP-C₂₋₁₄ antibodies confirming the presence of two proteins at 70-kDa.

FIG. 6A represents a scanned image of MI procedure performed on test rats showing the passage of a 6-0 silk suture through the left ventricular apex to position the heart and help identify the LAD coronary artery. To induce MI, another 6-0 suture was used to ligate the LAD about four to five micrometers inferior of the left atrium along the right pulmonary outflow tract.

FIG. 6B represents a MI heart showing the presence of severe left ventricular infarct, compared to sham hearts.

FIG. 6C represents a M-mode echocardiograms showing loss of wall motion and left ventricular dilation in post-MI hearts, compared to sham hearts.

FIG. 6D represents graphs of the results of trials showing that rat hearts with MI showed significant reduction in wall thickness, decreased fractional shortening, increased end systolic volume and decreased ejection volume, compared to sham hearts.

FIG. 6E represents cross-sections of sham rat hearts (×2) stained with hematoxylin and eosin (H&E) and trichrome (Trich.) showing normal and centrally located nuclei in intact myocytes (×20) and rat hearts with MI showing nucleomegaly, loss of cross striations in myocytes, loss of cardiomyocyte boundary, infarct zone, wavy fibers, neutrophilic infiltration, and degeneration of myocytes in histopathological changes (×20).

FIG. 7 represents scanned images of light and electron microscopic (EM) analyses of rat hearts.

FIG. 8 represents SDS-PAGE analyses of tissue homogenate from naïve, sham and infarct hearts showing the protein profile of anterior wall (AW) and posterior wall (PW) of MI heart, compared to naïve and sham controls.

FIG. 9A represents Western blot analyses of tissue homogenates from naïve, sham and infarct hearts showing the proteolytic pattern of cMyBP-C using rabbit anti-cMyBP-C₂₋₁₄ antibodies and the release of 40-kDa fragments in the anterior wall (AW) and posterior wall (PW) from an MI heart, compared to naïve and sham controls. NS (70-kDa) and glyceraldehyde phosphate dehydrogenase (GAPDH) were used as loading controls.

FIG. 9B represents a graph showing significant reduction (15±3.0%) of total cMyBP-C observed in AW of MI heart compared to sham hearts.

FIG. 9C represents a graph showing a significant increase in 40-kDa fragments in the AW region of MI hearts.

FIG. 9D represents phosphorylation levels of Ser-273, Ser-282 and Ser-302 determined in naïve, sham and infarct heart tissue by Western blots using phospho-specific antibodies. GAPDH was used as a loading control and normalized with total cMyBP-C.

FIG. 9E represents a graph showing a significant reduction of cMyBP-C phosphorylation in the AW of MI hearts, compared to the AW of sham controls and a significant increase in cMyBP-C phosphorylation levels in the PW of MI hearts, compared to the sham controls.

FIG. 9F represents Western blot analyses used to determine total levels of cMyBP-C and phosphorylation levels of cMyBP-C in non-failing and failing human hearts using total tissue homogenate. The analyses show the presence of a 40-kDa N-terminal fragment in the failing samples, compared to NF samples.

FIGS. 9G-H represent graphs showing phosphorylation levels that are significantly reduced and directly related to the increased release of 40-kDa peptides in the failing samples.

FIG. 10A represents Western blot analyses using 10 μg of total proteins from the AW of the MI rat heart tissue (MI AW) and 6 hrs effluent from FIG. 1 (ERM) with rabbit anticMyBP-C₂₋₁₄ antibodies showing that the 40-kDa peptide is the predominant small fragment of cMyBP-C N-terminal region and released easily into the effluent during MI. The 40-kDa fragments were partially purified from rat heart tissue.

FIG. 10B represents partial purified fragments that were resolved on a longer SDS-PAGE (20 cm height) and silver stained using SilverQuest (Invitrogen). Results show the presence of two fragments (F1 and F2) at the 40-kDa region.

FIG. 10C represents Western blot analyses using the partially purified 40-kDa fractions with rabbit anti-cMyBP-C₂₋₁₄ antibodies confirmed the presence of two fragments (F1 and F2) at 40-kDa. Both fragments were individually cut out from the silver stained SDS-PAGE gel and used for MALDI-TOF to identify mass and amino acid sequences.

FIG. 10D represents a MALDI-TOF MS profile of tryptic peptides from fragment 1 and 2. Red=cMyBP-C tryptics; Black=trypsin auto-digestion; Blue=cMyBP-C peptide NOT in Fragment 2.

FIG. 10E represents a MALDITOF/TOF MS sequence confirmation for fragment 1 and 2.

FIGS. 11A-D represent a Western blot analyses of total proteins from the anterior wall (AW) and posterior wall (PW) naïve, sham and infarct hearts with rabbit anti-cMyBP-C^{C5} (A) and anticMyBP-C^{C8-C9} antibodies. Results confirm that total cMyBP-C levels were significantly reduced (15±3%) in the AW region of MI hearts using C5 (B) and C8-C9 (D) antibody.

FIG. 12A represents Western blot analyses using mouse monoclonal anti-cMyBP-C^{CO} antibody on five to fifteen μg of albumin-depleted plasma samples from sham and I-R rats.

FIG. 12B represents an immunoprecipitation assay using sham and I-R plasma samples of rats with mouse monoclonal anti-cMyBP-C^{CO} antibody confirming the presence of cMyBP-C in the plasma. (D: plasma samples, IP: immunoprecipitated samples, N: Negative control, P: Positive control)

FIG. 12C represents a sensitivity analysis of N′-specific antibodies by Western blot analyses: (i) SYPRO Ruby-stained gel, (ii) Western with rabbit polyclonal anticMyBP- C_{C0} antibody and (iii) Western with mouse monoclonal anti-cMyBP-C^{C0} antibody.

FIG. 12D represents a graph using recombinant 40-kDa peptides for the sandwich ELISA.

FIGS. 12E-F represent graphs showing plasma levels of cMyBP-C in sham and MI rats and plasma levels of cTnI in sham-operated and MI rats. *P<0.001, sham (n=12) vs. patients (n=9).

FIG. 12G represents Western blot analysis of plasma samples of human control and patients by using mouse monoclonal anti-cMyBP-C^{CO} antibody, demonstrating the presence of cMyBP-C in blood post-MI.

FIGS. 12H-I represent graphs showing plasma levels of cMyBP-C in human control and patients and plasma levels of cTnI human in control and patients with MI. Values are mean±S.E.M. *P<0.001, control (n=26) vs. patients (n=15).

FIG. 13 represents Western blot analyses of total plasma proteins from sham and MI rat hearts with rabbit anti-cMyBP-C^{C0-C1} antibodies. Fifty μg of total plasma samples were loaded onto 4 to 15% SDS-PAGE and Western blot was performed using rabbit anticMyBP-C^{C0-C1} antibodies. Results show the presence of full-length cMyBP-C and its cleaved 40-kDa fragments in the MI plasma samples.

### DETAILED DESCRIPTION OF THE INVENTION

The present invention will be described in terms of methods and assays for risk prediction, diagnosis, and analysis of myocardial infarction (MI), myocardial failure, and reduced cardiac function. In one aspect of the present invention, the potential risk of MI is determined by DNA analysis of cardiac myosin binding protein-C. In another aspect of the present invention, detection of cardiac myosin binding protein-C proteins and its peptides, phosphorylation status and autoantibodies in human body fluids or tissue samples is used as a predictor/indicator to detect MI and determine its severity.

Cardiac myosin binding protein-C (cMyBP-C) is a 140-kDa sarcomeric thick filament protein and myosin stabilizer that is involved in regulating sarcomeric structure and function in the heart. Increasing evidence suggests that cMyBP-C plays a critical role in regulating myosin function and cardiac contraction. Given that mutations in this protein have been linked to familial hypertrophic cardiomyopathy in more than 60 million people worldwide, it is clinically urgent to fully elucidate the function of cMyBP-C. This protein comprises 2% of the total contractile protein in the heart, and it belongs to the intracellular immunoglobulin superfamily which is composed of repeating domains of immunoglobulin and fibronectin type-3, both of which are hydrophilic in nature. cMyBP-C phosphorylation at Ser-273, Ser-282 and Ser-302 regulates myocardial function and confers resistance to proteolysis and protection against MI. On the other hand, the degradation of cMyBP-C during MI correlates with contractile dysfunction. cMyBP-C is a substrate for calpains that cleave cMyBP-C during MI, resulting in the release of a 40-kDa fragment, a polypeptide that could cause pathogenic cardiac muscle damage.

An association has been reported between a 25-nucleotide deletion (AGCCTGGATG GCTTCCCTCC CTCTC) in the cMyBP-C gene and an increased risk of heart failure in South Asian populations and inherited in 1% of world populations. The 25-nucleotide deletion is located in the cMyBP-C at the carboxyl terminus.

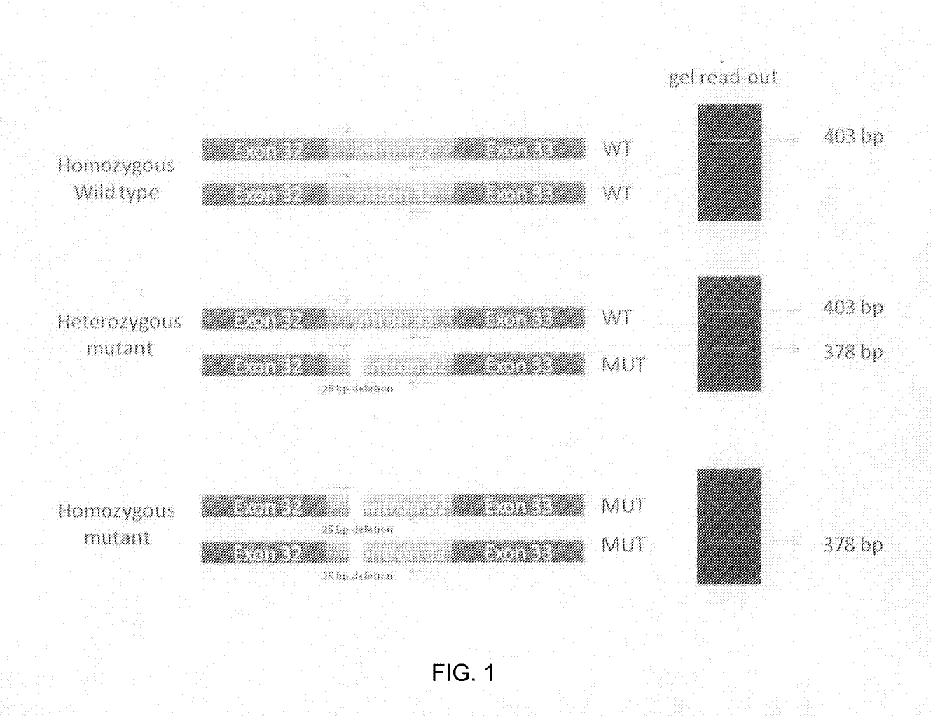

In a preferred aspect of the present invention, detection of the 25-nucleotide deletion may be used as a clinical method of predicting an increased risk of MI in patients. A blood sample may be collected from subjects for genomic DNA extraction. The presence of cMyBP-C 25-nucleotide mutation may then be identified by techniques conventional to the art. An example of such a technique is polymerase chain reaction (PCR). Acceptable primers for detection of the 25-nucleotide deletion are forward primer: 5′-GTT TCC AGC CTT GGG CAT AGT C-3′ and reverse primer: 5′-GAG GAC AAC GGA GCA AAG CCC-3′. PCR amplification using this primer pair results in either a normal 403 bps band or a mutation-specific 378 bps band, as represented in FIG. 1. Such an assay can be embodied in test kits that may use fluorescence probes to determine the presence or absence of the mutation by real-time PCR. Using this technique, physicians may be able to better predict and counsel patients that have an increased risk of MI.

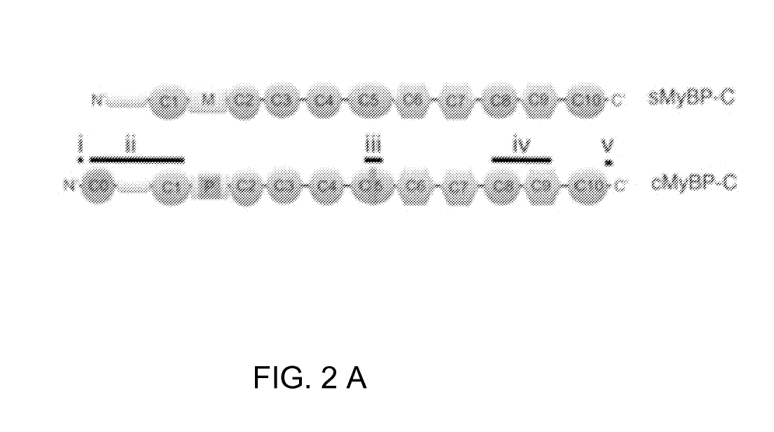

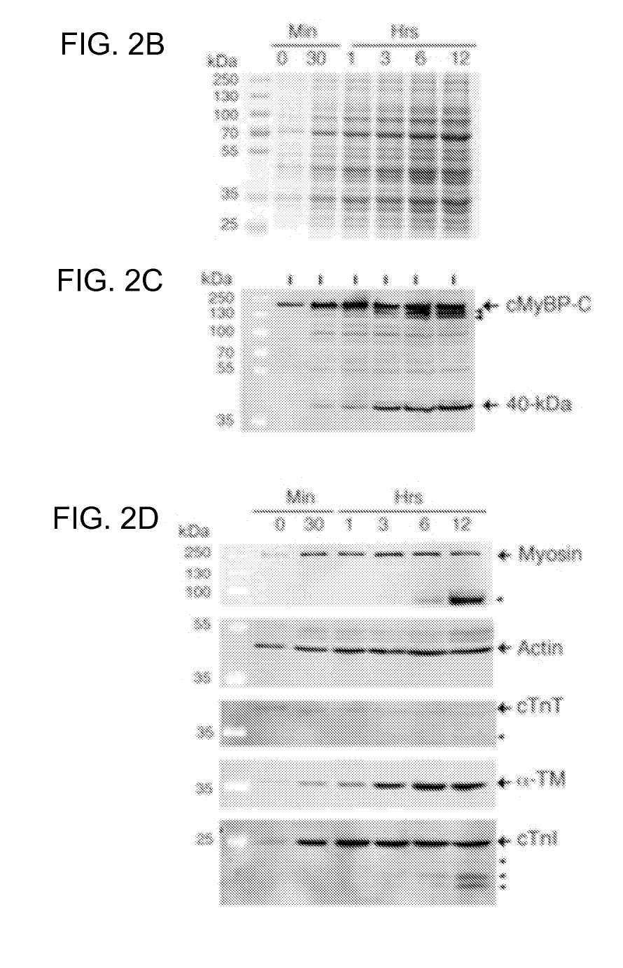

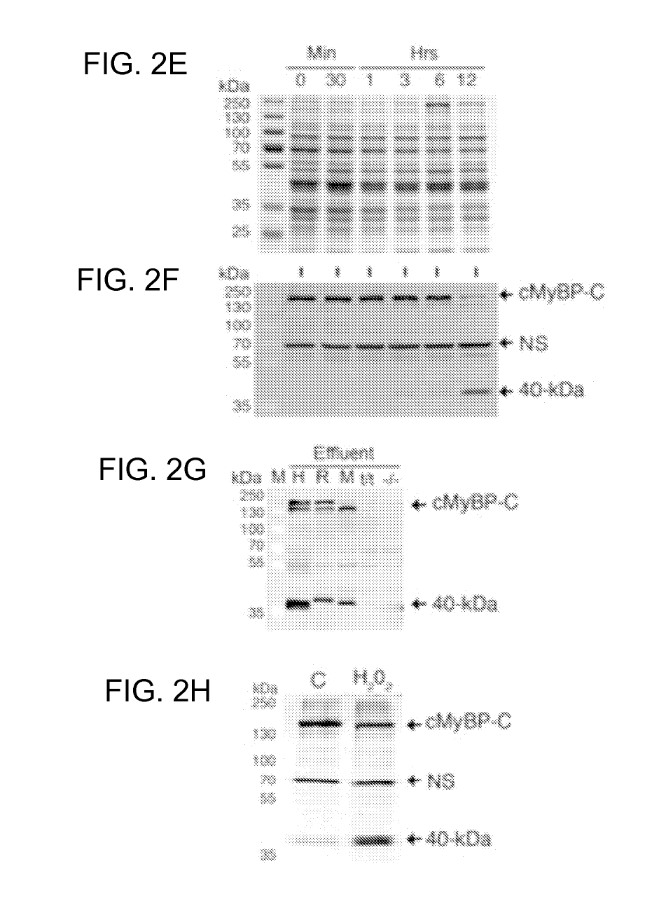

Once a patient has suffered MI, it is important that physicians are able to quickly and accurately diagnose the ailment and determine its severity. In one embodiment of the present invention, MI can be diagnosed by quantifying the presence of cMyBP-C in patients. cMyBP-C is expressed exclusively in heart atria and ventricles, whereas the two skeletal MyBP-C (sMyBP-C) isoforms are specific to skeletal muscles. cMyBP-C differs from sMyBP-C isoforms by having an extra C0 domain with 101 residues at the N′-region, a phosphorylation motif (m-motif) which contains an additional nine unique residues and three phosphorylation sites, and a 28-residue insertion in a C5 domain as represented in FIG. 2A. The N′-region is floating and partially interacts with myosin and actin, while the C′-region is critical for protein anchorage and interaction with myosin and titin. To determine if cMyBP-C could be used as a marker for determining the severity of damage to the heart post-MI, a series of trials were preformed that involved testing of rodents and human samples.

The trials on rodents involved ten-week old male Sprague Dawley rats. Acute myocardial infarction was induced on rodents, which hereinafter are referred to as ischemia-reperfusion (I-R)-injured rats. Controls included naïve rats, which were not operated on and sham rats, which underwent surgery but did not undergo MI. Three days post-MI, left ventricular structure and function was measured in MI, naïve and sham animals by non-invasive M-mode echocardiography, and blood and tissue samples were collected for analyses. For the human trials, plasma samples were collected from normal controls and patients admitted to the Scott & White Hospital, Temple, Tex., who were diagnosed with MI based on the electrocardiogram findings and elevations in cTnI levels (>5.0 ng/ml). Non-failing and failing heart muscle tissue samples were obtained from the tissue repository at the Cardiovascular Institute of Loyola University Chicago Medical Center, Maywood, Ill.

cMyBP-C phosphorylation by protein kinase A (PKA) regulates myocardial function and confers resistance to proteolysis and protection against MI. However, the degradation of cMyBP-C during MI correlates well with contractile dysfunction. To determine whether cMyBP-C is an easily releasable myofilament protein during necrosis, left ventricular (LV) heart tissue from wild-type rats was incubated with phosphate buffered saline (PBS) at 37° C. for different time points. During the necrotic period, all releasable proteins in the ventricle tissue were expected to release slowly into the PBS solution. SDS-PAGE analyses confirmed that the release of total heart proteins was increased over time as represented in FIG. 2B. To determine the release of full-length cMyBP-C and its proteolytic fragments into the PBS effluent, Western blot analysis was performed with N′- specific rabbit anti-cMyBP-C₂₋₁₄ antibodies, the results of which are represented in FIG. 2C. This antibody recognized full-length cMyBP-C and its N′-fragments. Results show that cMyBP-C was released into the effluent from one second to twelve hours. At the one second time point, intact cMyBP-C was present, followed by an increased release of full-length and C′-truncated cMyBP-C starting at 30 min. Specifically, 40-kDa N′-terminal fragments of cMyBP-C were predominantly released starting from one hour. Furthermore, release of cMyBP-C and its fragments increased in a time-dependent manner. In comparison, other cardiac sarcomeric proteins, such as myosin, actin, cTnI, cTnT and α tropomyosin (α-TM), were also found to be released into the effluent as represented in FIG. 2D. Results confirmed that cTnI, cTnT and α-TM were significantly increased over time. The release of cMyBP-C fragments in vitro was identified using antibodies that were raised against C5, C8-C9 and C10 domains. The results are represented in FIG. 3 and show that cMyBP-C is degraded at multiple sites.

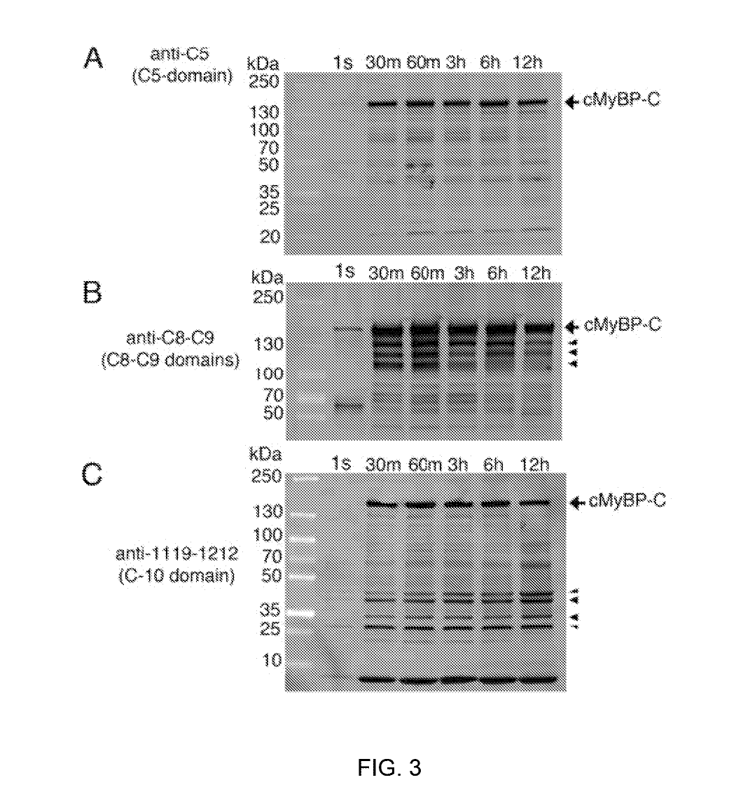

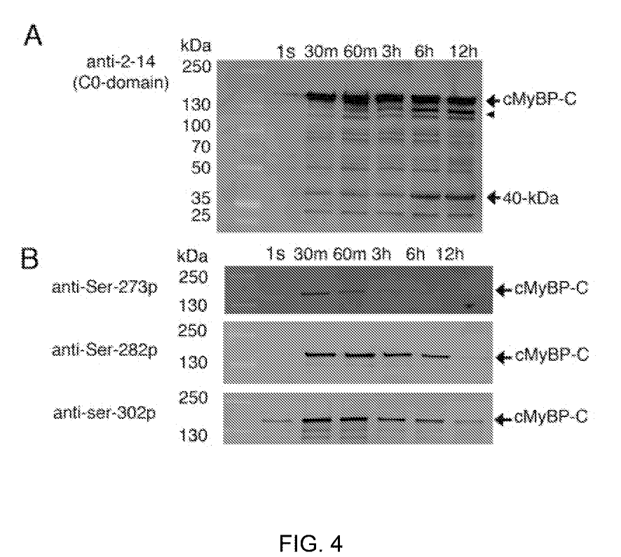

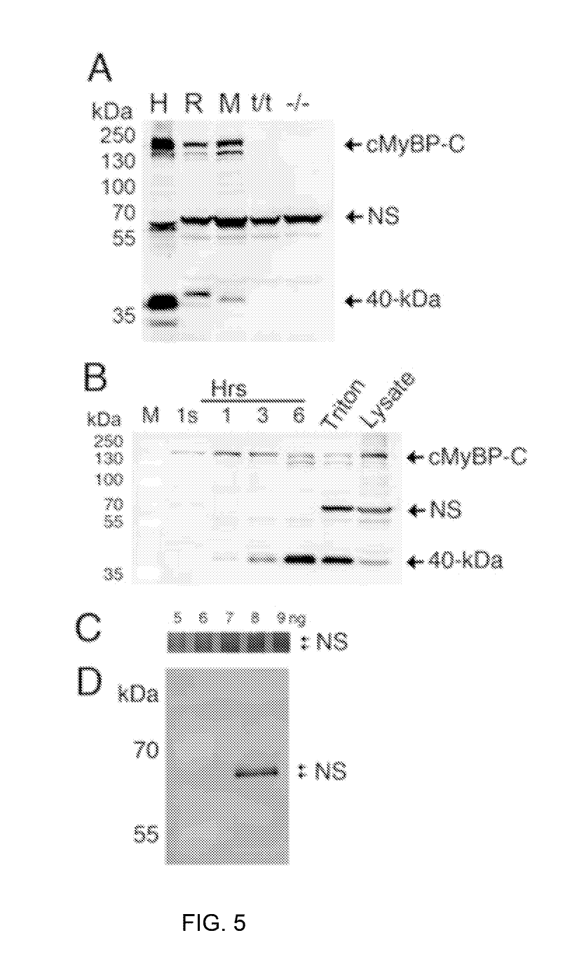

Next, to examine how much cMyBP-C was left in the tissue, the tissue was homogenized in RIPA buffer and analyzed by SDS-PAGE and Western blot with anticMyBP-C₂₋₁₄ antibody, the results of which are represented in FIGS. 2E and 2F respectively. The results show that full-length cMyBP-C was present at all time points and that its level was reduced only at the twelve hour time point, suggesting that cMyBP-C degradation may take place only after release of full-length cMyBP-C in the PBS effluent. Further, it was confirmed that all the released cMyBP-C in the effluent was dephosphorylated at Ser-273, Ser-282 and Ser-302 over time, as evident from FIG. 4B. The 40-kDa fragments were the smallest of the predominantly released N′-fragments in human and mouse hearts, as represented in FIG. 2G, but not in homozygous cMyBP-C null and knockout mouse hearts. The predominant 40-kDa proteolytic fragments were confirmed to be significantly released during hypoxic stress using neonatal rat ventricular cardiomyocytes compared to control cells, the results of which are represented in FIG. 2H. During periods of ischemia and necrotic transitions, the rate of sarcomeric protein degradation increases in association with activation of proteolytic enzyme activity. Calpain is a calcium-activated cysteine protease that regulates myofilament proteins, and plays a major role in myofibrillar degradation, and it is activated during ischemic injury. cMyBP-C is a substrate for calpains. Therefore, during muscle degradation, it was expected that activated calpains would cleave cMyBP-C and other sarcomeric proteins, such as cTnI and cTnT. Unexpectedly, cMyBP-C was predominantly released into the effluent, either as intact protein or as cleaved fragments. In addition, the rate of cMyBP-C diffusion into the effluent, either in full-length or fragments, was increased rapidly over time. Altogether, these results suggest that cMyBP-C is an easily releasable sarcomeric protein and that the 40-kDa fragments are the predominant small N-terminal fragments released during cMyBP-C proteolysis.

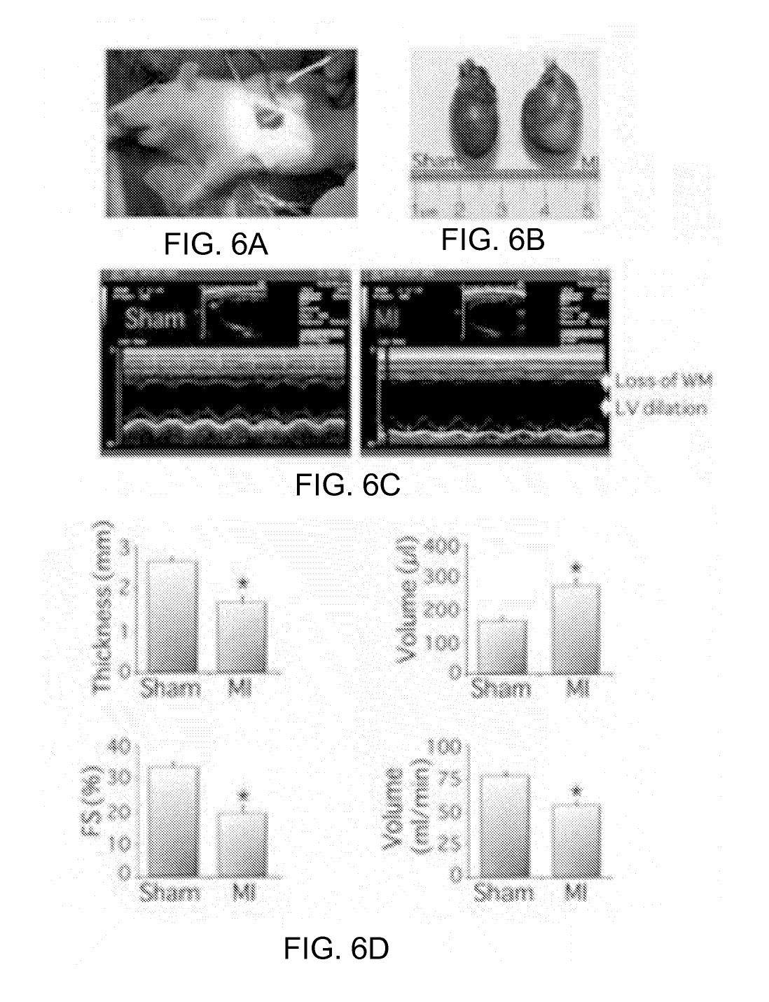

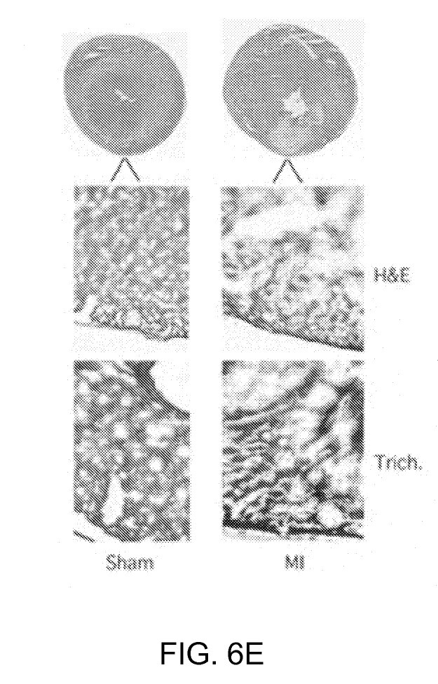

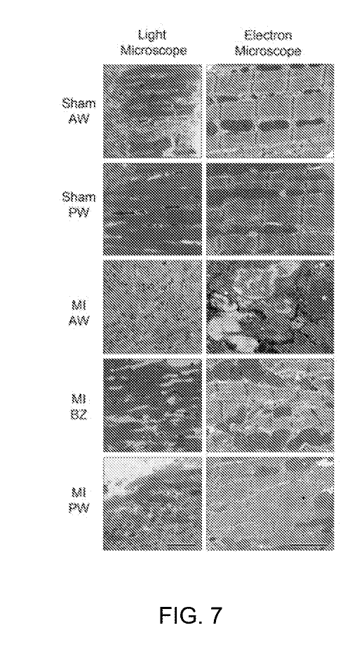

During ischemia, the thick and thin sarcomeric proteins are modified, disorganized and degraded, thereby altering contractile function. In order to study the phosphorylation and degradation pattern of cMyBP-C in a ischemic region of MI hearts compared to an undamaged region of the heart, MI was induced in rats with sham-operated animals as controls. Examples of the rats and hearts are represented FIGS. 6A and 6B. LV function was measured three days after MI by echocardiography to define the characteristic features of post-MI injury. Functional data show that left anterior descending coronary artery occlusion resulted in hypertrophied hearts with loss of LV wall motion and LV dilation, as represented in FIG. 6C. In addition, reduced wall thickness, decreased fractional shortening, increased end systolic volume and decreased ejection volume were apparent three days post-MI, compared to sham-operated animals, as represented in FIG. 6D. Morphological and histopathological analyses revealed the presence of infarct area in MI hearts, necrosis in the ischemic region and extended fibrosis, compared to sham rat hearts, as represented in FIG. 6E. Light and electron microscopic analyses, as represented in FIG. 7, showed disorganized myofibrillar structure in the infarcted anterior wall (AW) region, compared to the border zone and posterior wall (PW), suggesting that MI results in severe damage of myofibrillar structure. The data showed the presence of reduced cardiac function, significant necrosis and disorganized myofibrillar structure at three days post-MI.

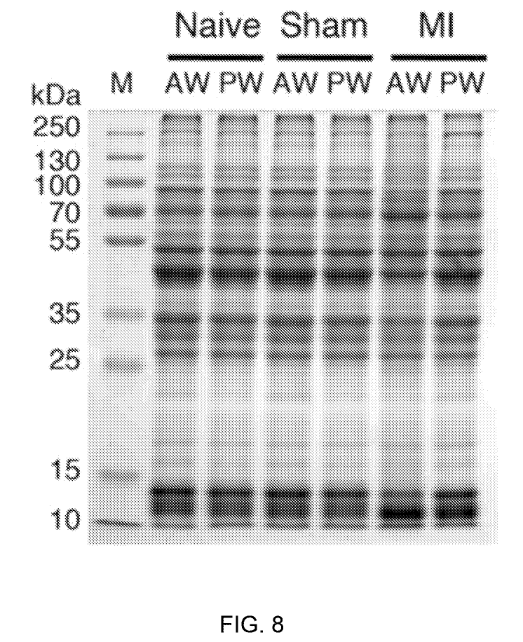

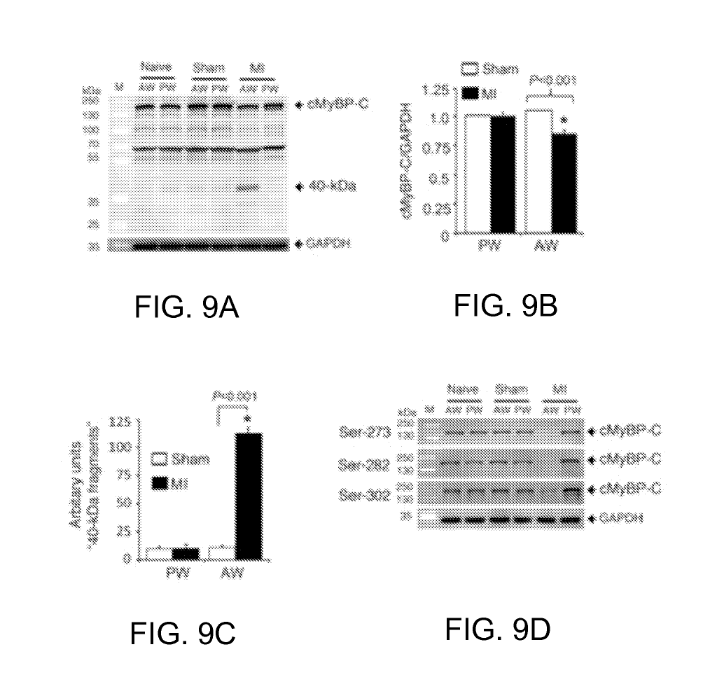

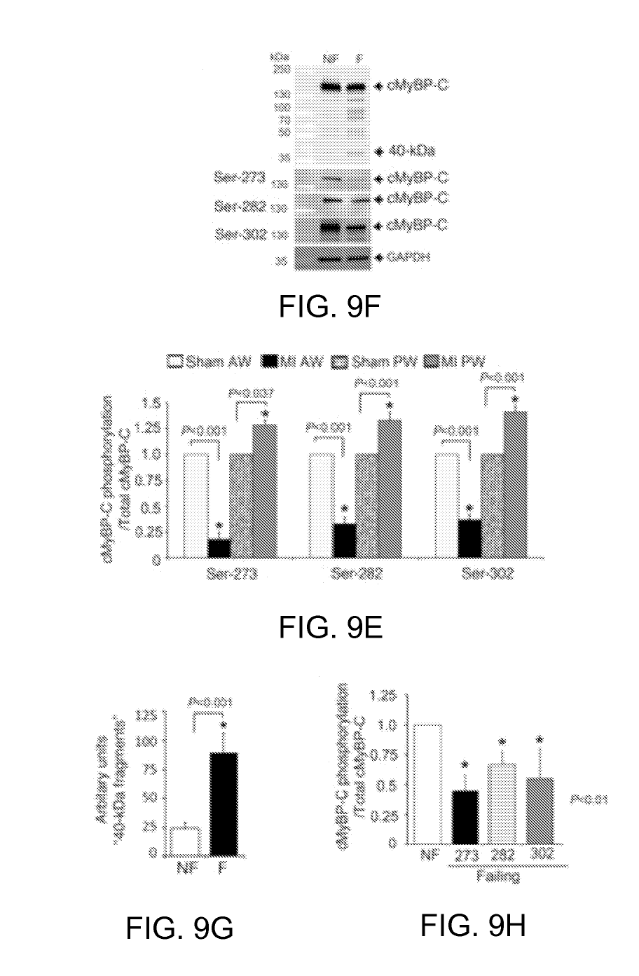

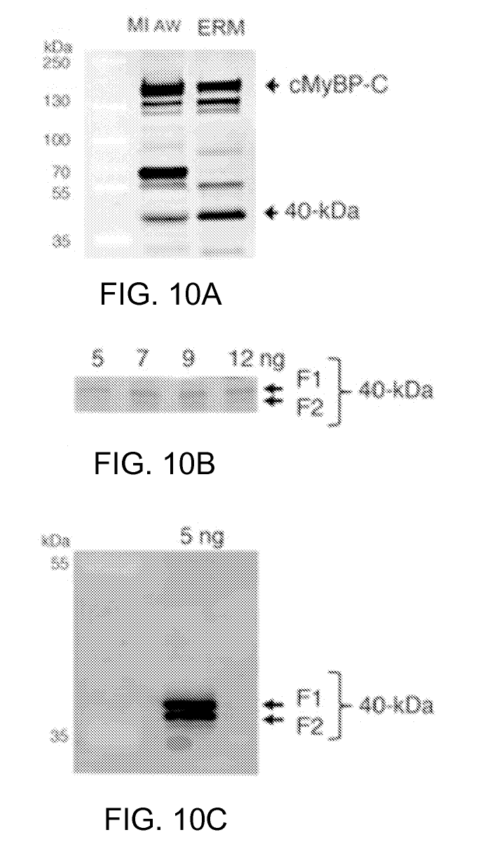

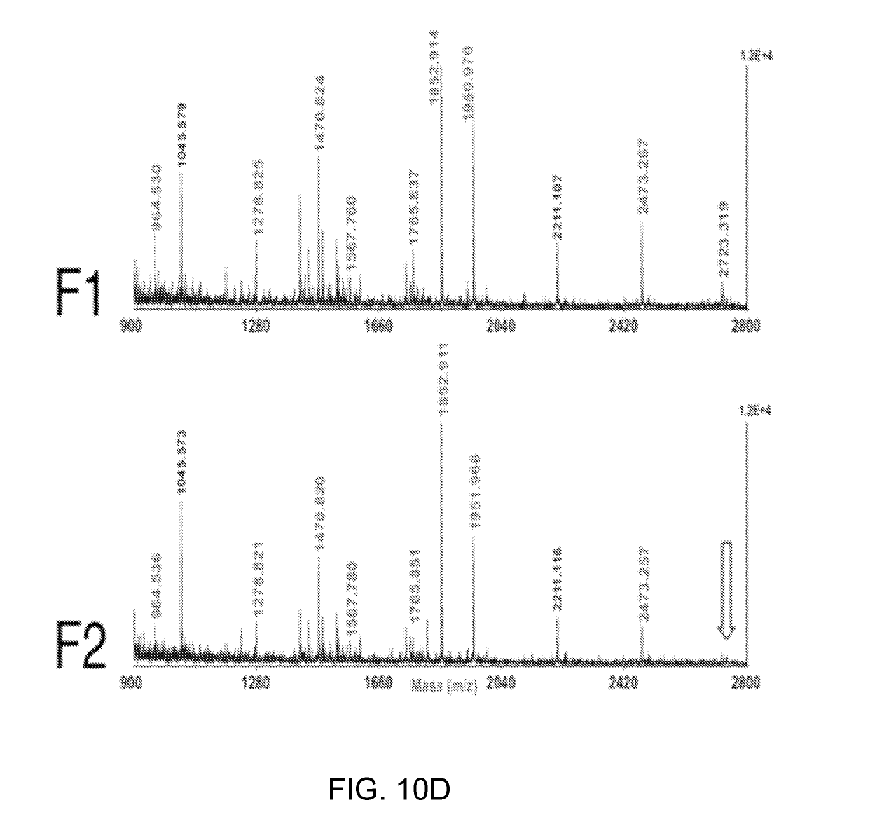

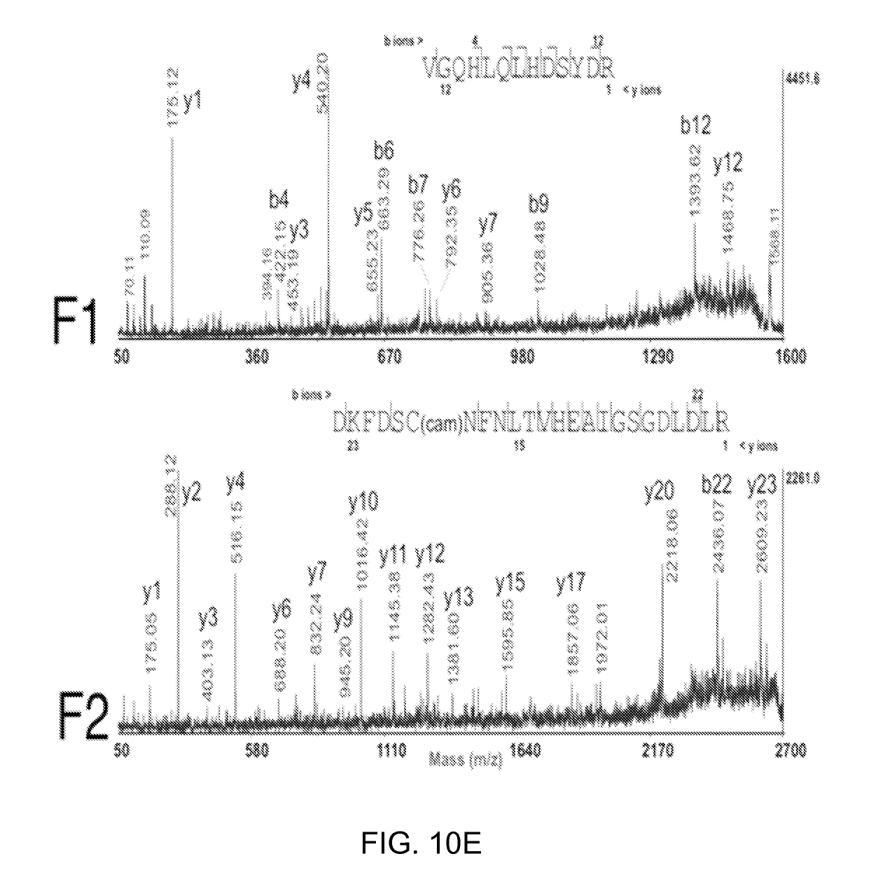

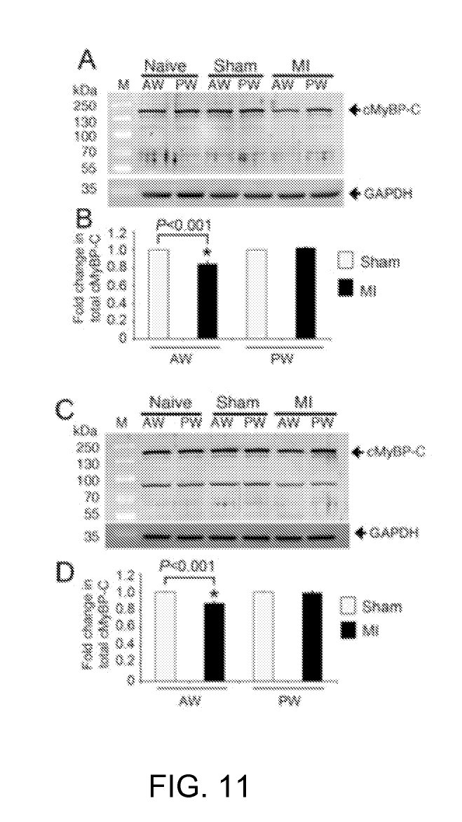

SDS-PAGE analyses determined the altered total protein profile in the ischemic AW region in response to MI, as compared to the remote PW region and controls, as represented in FIG. 8. The level of cMyBP-C and its degradation profile was determined using total proteins from LV, AW, and PW regions of naïve, sham and MI hearts by Western blot analyses, using C0, C5, C8-C9 and C10 domain specific anti-cMyBP-C antibodies. As a result, it was possible to measure the level of cMyBP-C post-MI. Using C0-domain-specific anti-cMyBP-C₂₋₁₄ antibodies, it was determined that total cMyBP-C was significantly reduced in the AW region of the MI hearts, compared to the PW of the same hearts and controls, as represented in FIGS. 9A and 9B. In addition, the appearance of the 40-kDa fragment was evident and significantly increased in the AW region of MI hearts, represented in FIGS. 9A and 9C, confirming that the release of 40-kDa fragments is specific to necrotic LV tissue. Next, 40-kDa fragments were partially purified and separated using high resolution SDS-PAGE. The results show that there were two fragments, F1 and F2, at the 40-kDa position, and they were identified as rat cMyBP-C, as represented in FIGS. 10A through 10E. Strikingly, F1 and F2 fragments were cleaved in the m-motif, close to the three phosphorylation sites. Furthermore, using C5, C8-C9, and C10 domain-specific anti-cMyBP-C antibodies, it was determined that the total level of cMyBP-C was significantly reduced in the AW region of the MI hearts, compared to controls, as represented in FIG. 11. These data suggest that cMyBP-C undergoes proteolysis during MI, which results in reduction of total cMyBP-C level and the release of 40-kDa fragments.

To determine whether dephosphorylation of cMyBP-C is directly associated with the degradation and release of its 40-kDa fragments in the AW region post-MI, the phosphorylation levels of Ser-273, Ser-282 and Ser-302 in naïve, sham-operated and infarcted hearts in the AW and PW regions were quantified using site-specific phospho antibodies, the results of which are represented in FIG. 9D. In the AW region of the MI heart, results showed a significant decrease in phosphorylation levels of Ser-273, Ser-282 and Ser-302, as represented in FIG. 9E, when normalized with total cMyBP-C, suggesting that dephosphorylation of cMyBP-C is associated with cMyBP-C degradation. As a compensatory effect of AW ischemia, the PW region of MI hearts showed a significant increase in cMyBP-C phosphorylation levels at Ser-273, Ser-282 and Ser-302 sites, without showing release of the 40-kDa fragments. Human failing heart samples were then examined to determine whether cMyBP-C phosphorylation is decreased and associated with its degradation compared to non-failing heart samples. Results showed the presence of 40-kDa fragments in six out of nine samples. Although there was a trend in the reduction of total cMyBP-C in the failing samples, it was not significant. However, failing samples showed significant decrease in phosphorylation levels of Ser-273, Ser-282 and Ser-302. Altogether, the data demonstrate that cMyBP-C undergoes severe degradation during MI and in failing hearts. Therefore, the release of 40-kDa fragments are believed to directly associate with cMyBP-C dephosphorylation.

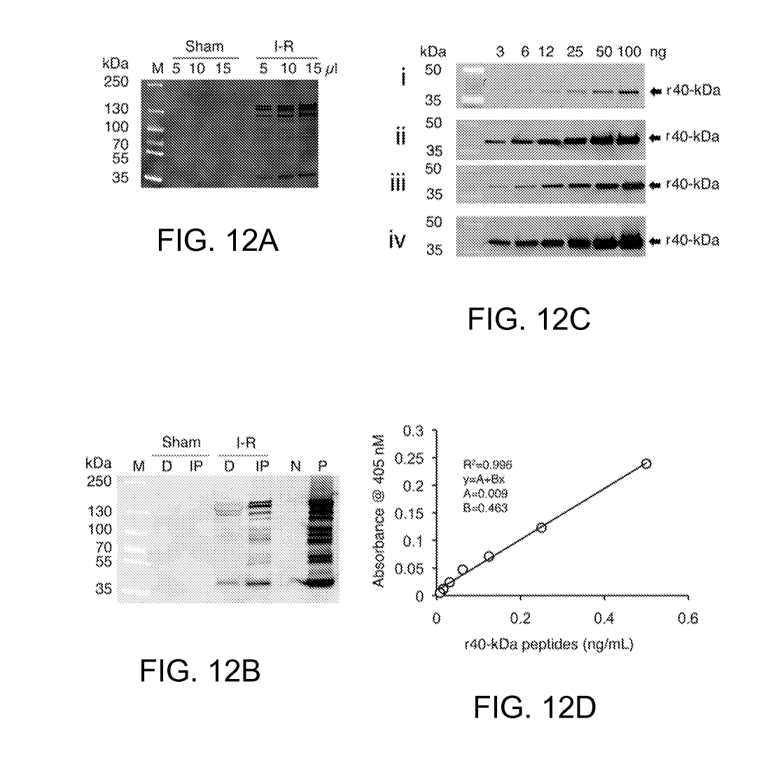

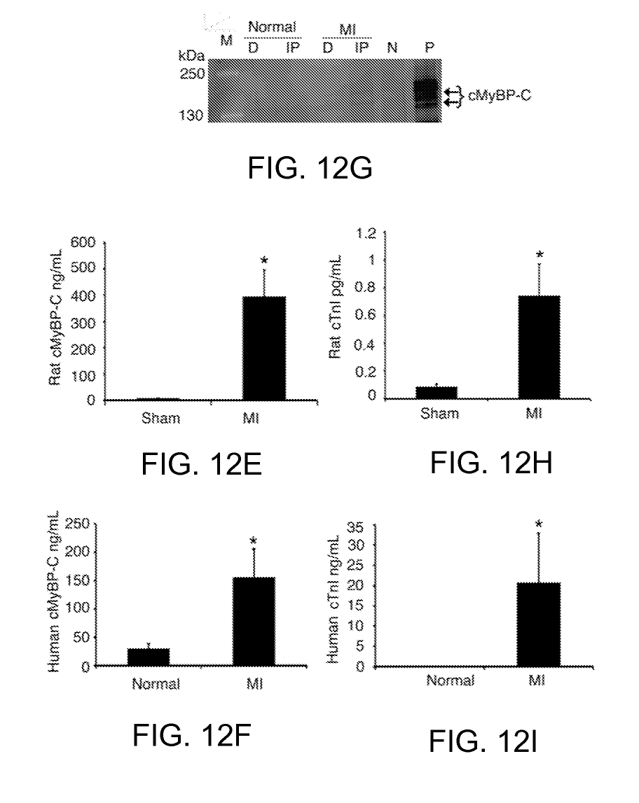

Some of the consequences of AMI are increased diastolic Ca2+ concentration, activation of proteases that target contractile proteins, such as cTnT and cTnI, leading to deterioration of myocardial contractile function, ventricular dilation and eventually heart failure, and the appearance of contractile proteins in the circulation. To determine if cMyBP-C is released into the circulatory system as a consequence of MI, the plasma levels of cMyBP-C in sham-operated and MI animals were determined by Western blot analyses and sandwich ELISA by using mouse monoclonal anti-cMyBP-C^{C0} and rabbit anticMyBP-C^{C0-C1} antibodies, the results of which are represented in FIGS. 12C and 12D.

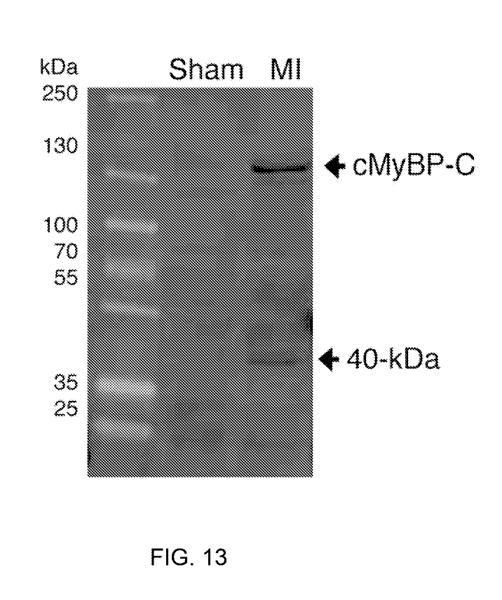

First, Western blot analyses using samples from sham and ischemia-reperfusion (I-R)-injured rats was conducted. Results showed that cMyBP-C levels in sham rats were undetectable, as represented in FIG. 13, whereas cMyBP-C and its degraded fragments were significantly increased in the plasma of rats with I-R injury, as represented in FIG. 12A. The presence of intact cMyBP-C and its fragments in the plasma samples of I-R rats was further confirmed by immunoprecipitation, as represented in FIG. 12B. Next, the presence of cMyBP-C and its fragments was confirmed using the plasma samples of post-MI rats. Sandwich ELISA results show that cMyBP-C in the MI rat plasma samples was significantly increased (450±50 ng/ml) compared to sham rats (5.6±0.5 ng/ml), as represented in FIG. 12E. This was higher than cTnI levels in the MI plasma samples (738 pg/ml), compared to sham rats (80±0.5 pg/ml), as represented in FIG. 12F, suggesting that increased cMyBP-C levels in the circulatory system could be a potential biomarker for detecting MI. Next, cMyBP-C levels in the plasma samples of patients with MI was analyzed.

To determine the level of cMyBP-C in the plasma human samples, sandwich ELISA was established. Frozen plasma samples stored at −80° C. were brought to room temperature. Completely thawed samples were centrifuged to remove precipitated proteins. Then the plasma was diluted four times with PBS. One hundred μl of 4× diluted plasma samples were added to the capture antibody coated 96 well ELISA plates. Mouse monoclonal cMyBP-C^{C0} antibody (Santa Cruz, clone E7) was used for coating the ELISA plates overnight as a capture antibody and Rabbit polyclonal cMyBP-C^{C0} antibody was used as a detection antibody. Both antibodies were able to recognize the intact protein and any N-terminus fragments of cMyBP-C. Secondary Donkey anti-Rabbit IgG antibody conjugated with horseradish peroxidase (HRP) (Santa Cruz, Cat. No. SC2305) was used for detecting Rabbit polyclonal anti-cMyBP-C^{C0} antibody and ABTS was used as substrate for HRP. Green color development was measured at 405 nm in a 96 well plate ELISA reader (VersaMax ELISA Microplate reader, Molecular Devices). ELISA plates (BDFalcon Cat. No. 353911, Franklin Lakes, U.S.A) were coated with 1:1000 dilution of mouse monoclonal anti-cMyBP-C^{C0} antibodies in 100 μl of PBS and closed with lid (BD-Falcon Cat. No. 353913, Franklin Lakes, U.S.A) and incubated for 16 hours at 4° C. After 16 hours incubation, the plate was washed three times with 150 μl of PBS buffer containing 0.01% Tween 20 using an automated microplate washer (Bio-Rad, ImmunoWash 1575). Then the antibody coated wells were blocked with 1% blocking solution (Cat. No. 11921681001, Roche Diagnostics, Indianapolis, Ind., USA) in PBS for one hour at room temperature. The wells were washed 3 times with 150 μl of Phosphate Buffered Saline with Tween 20 (PDST) using the automated microplate washer. Recombinant 40-kDa, 1-272 residues of N-terminal cMyBP-C, was used for the standard graph. Standard protein and the 4× diluted plasma samples in 100 μl volume was added to the wells and incubated at room temperature for 1 hour. Then the plate was washed 3 times with 200 μl PBS-T. After draining the droplets carefully, 100 μl of 1.0 μg/ml of Rabbit polyclonal anti-cMyBP-C^{C0} was added and incubated at room temperature for 1 hour. Wells were washed again 3 times with 200 μl of PBST. For detection, 100 μl 1:1000 diluted anti Goat-HRP was added to each well and incubated for 30 minutes at room temperature. The plate was washed again using PBST and the substrate ABTS (2,2′-azino-bis(3-ethylbenzothiazoline-6-sulphonic acid)) was added and incubated at room temperature. Development of green color was measured at 450 nm. A standard graph was made using the known concentration of recombinant 40-kDa peptides. Plasma samples were collected from 16 AMI patients with an average age of 66±4 yrs and a left ventricular ejection fraction (LVEF) of 39±2% at 42±6 hrs after the first MI symptoms appeared. Blood glucose levels were 185±31 mg/mL with a creatinine level of 1.16±0.114 mg/dL. Immunoprecipitation analyses confirmed the presence of cMyBP-C in the plasma samples of patients with MI, as represented in FIG. 12G, but not in control samples. cMyBP-C and cTnI levels in the plasma samples of 26 healthy volunteers were 0 ng/mL and 18.2±0.5 ng/mL, respectively. Strikingly, the cMyBP-C plasma levels were greater than the cTnI levels, 210±57 vs. 30±9 ng/mL, suggesting that the increased level of cMyBP-C in the circulatory system is a sensitive signal of post-MI.

In the above trials, the level of full-length and fragmented cMyBP-C in the plasma was determined using a rat MI model 72 hrs post-MI. Based on the results in this trial and available preliminary data, it was concluded that the presence of cMyBPC in the circulatory system is a sensitive signal of MI. During the early stage of MI, intracellular pH slowly drops, phosphatases are activated, and transient increase in Ca2+ activates proteases like calpains that target myofilament proteins. In addition, MI leads to necrosis, which is accompanied by the activation and release of proteolytic enzymes such as calpains from lysosomes. Because cMyBP-C is a substrate for calpains, MI could induce significant degradation of cMyBP-C. After MI, contractile dysfunction is accompanied by calpain activities and cMyBP-C degradation, compared to control. Importantly, cMyBP-C is easily dissociated at around pH 6.0, compared to other sarcomeric proteins, such as myosin and troponins I and T. cMyBP-C is cleaved post-MI and appears in the blood with a proteolytic pattern similar to that seen in vitro and in vivo. From these trials, it is believed that variable magnitudes of intact cMyBP-C and its proteolytic fragments will continue to circulate in the blood, depending on the time of MI onset, size of infarct region, and rate of reperfusion in AMI patients. The in vitro and in vivo trials showed that the N-terminal regions of cMyBP-C were extremely susceptible to proteolysis and easily removed from the necrotic tissue that may be easily released into the blood. Taken together, this trial defines the proteolytic pattern of cMyBP-C post-MI, the dephosphorylation-dependent cMyBP-C degradation and a role for cMyBP-C released into the circulation as a biomarker for measuring the pathogenesis of MI. Overall, these data suggest that the plasma level of cMyBP-C is a specific, quantitative, and sensitive measure of MI.

The trials provided a framework for the development of methods for the quantitation of cardiac myosin binding protein-C protein and its autoantibodies in human biological fluid. Because cMyBP-C is very susceptible to proteolysis and undergoes rapid degradation after necrosis and release in higher concentration into the blood, the use of a mixture of different domain-specific antibodies, particularly monoclonal antibodies, to determine the sensitive and reproducible detection of cMyBP-C and its fragments in blood samples is believed to be necessary.

An assay for the quantitation of cardiac myosin binding protein-C, in accordance with a preferred aspect of this invention, uses a sandwich assay employing specific-antibodies for cardiac myosin binding protein-C using a mixture of cardiac myosin binding protein-C-specific domain antibodies that provides multiple advantages. A mixture of C0, C5 and C10 domain-specific antibodies has advantage over the use of individual antibody. An assay for quantitation of cardiac myosin binding protein-C in a human biological fluid may comprise, but is not limited to, incubating, sequentially or simultaneously, a sample of a human biological fluid and mixture of antibodies or antibody fragment specific for all or an antibody reactive portion of the cardiac myosin binding protein-C to detect cardiac myosin binding protein-C protein and its peptides or indicators in the human body fluid. The results of this assay may be confirmed with immunoprecipitation analysis.

An assay for the quantitation of cardiac myosin binding protein-C autoantibodies, in accordance with a preferred aspect of this invention, uses cardiac myosin binding protein-C specific peptides incubated with human body fluid. A mixture of C0, C5 and C10 domain-specific peptides has advantage over other combinations of the specific peptides. An assay for quantization of cardiac myosin binding protein-C autoantibody in a human biological fluid may comprise, but is not limited to, incubating, sequentially or simultaneously, a sample of human biological fluid and cardiac myosin binding protein-C domain-specific for all or an antibody-reactive portion of the cardiac myosin binding protein-C to detect cardiac myosin binding protein-C autoantibodies. The results of this assay may be confirmed with immunoprecipitation analysis.

These assays can be embodied in kits to detect and quantify cardiac myosin binding protein-C and its peptides, phosphorylation status, and autoantibodies in human body fluids or tissue as indictor/predictor of MI and heart failure. Such kits may comprise the cardiac myosin binding protein-C and its peptides, phosphorylation status, indicators and/or autoantibodies. The kits can comprise one or more sets of antibodies, polyclonal and/or monoclonal, for a sandwich format wherein the antibodies recognize epitopes on the full-length proteins, and one set is appropriately labeled or is otherwise detectable for each gene product or reference protein of interest. A kit for use in an enzyme-immunoassay may include an enzyme-linked antibody and a substrate for the enzyme. The enzyme can, for example, be linked to either an antibody specific to a protein of interest or to an antibody to such a specific antibody.

While the invention has been described in terms of certain embodiments, it is apparent that other forms could be adopted by one skilled in the art. Therefore, the scope of the invention is to be limited only by the following claims.

1. A method of predicting an increased risk of myocardial infarction and heart failure in a subject by detecting a 25-nucleotide deletion in a cardiac myosin binding protein-C gene in human body fluids or tissue, the method comprising:
collecting a sample of human body fluids or tissue from the subject for genomic DNA extraction; and
identifying the presence of cardiac myosin binding protein-C 25-nucleotide deletion.

2. The method according to claim 1, wherein the 25-nucleotide deletion is detected by polymerase chain reaction.

3. The method according to claim 1, further comprising the step of detecting the 25-necleotide deletion with a PCR-based kit, wherein the PCR-based kit comprises:
at least one forward primer set that provides a first detectable signal on the occurrence of amplification of cardiac myosin binding protein-C; and
at least one reverse primer set that provides a second detectable signal on the occurrence of amplification of the reverse compliment of cardiac myosin binding protein-C.

4. The method according to claim 3, wherein the forward primer of the PCR-based kit is 5′-GTT TCC AGC CTT GGG CAT AGT C-3′ and the reverse primer of the PCR-based kit is 5′-GAG GAC AAC GGA GCA AAG CCC-3′.

5. A method of diagnosing myocardial infarction, heart failure and reduced cardiac function in a subject by detecting cardiac myosin binding protein-C, its peptides, its phosphorylation status, and/or its autoantibodies in human body fluids or tissue, the method comprising:
collecting a sample of human body fluid or tissue from a subject; and then
detecting the presence of cardiac myosin binding protein-C, its peptides, its phosphorylation status, and/or its autoantibodies.

6. The method according to claim 5, further comprising the step of quantifying the cardiac myosin binding protein-C proteins, its peptides, its phosphorylation status, and/or its autoantibodies to determine the severity of the myocardial infarction.

7. The method according to claim 5, further comprising the step of analyzing the cardiac myosin binding protein-C proteins, its peptides, its phosphorylation status, and/or its autoantibodies by Western blot analyses.

8. The method according to claim 5, further comprising the step of analyzing the cardiac myosin binding protein-C proteins, its peptides, its phosphorylation status, and/or its autoantibodies by sandwich enzyme-linked immunosorbent assay.

9. The method according to claim 5, further comprising the step of analyzing the cardiac myosin binding protein-C proteins, its peptides, its phosphorylation status, and/or its autoantibodies by immunoprecipitation analysis.

10. A kit adapted to detect cardiac myosin binding protein-C, its peptides, its phosphorylation status, and/or its autoantibodies in human body fluids or tissue, the kit comprising:
an immunoassay solution; and
a means of detecting cardiac myosin binding protein-C, its peptides, its phosphorylation status, and/or its autoantibodies in human body fluids or tissue.

11. The kit according to claim 10, wherein the means of detecting cardiac myosin binding protein-C, its peptides, its phosphorylation status, and/or its autoantibodies in human body fluids or tissue is at least one antibody that binds to cardiac myosin binding protein-C, its peptides, and/or its autoantibodies.

12. The kit according to claim 11, wherein the at least one antibody is chosen from the group comprising C0, C5 and C10 domain-specific antibodies.

13. The kit according to claim 10, wherein the kit further comprises a calibrator, diluents, an assay plate and/or detection reagents.


SEQUENCE LISTING (6 sequences)

SEQ ID NO: 1 - protein, 210 aa, Homo sapiens
MADGSSDAAREPRPAPAPIRRRSSNYRAYATEPHAKKKSKISASRKLQLKTLLLQIAKQELEREAEERRGEKGRALSTRCQPLELAGLGFAELQDLCRQLHARVDKVDEERYDIEAKVTKNITEIADLTQKIFDLRGKFKRPTLRRVRISADAMMQALLGARAKESLDLRAHLKQVKKEDTEKENREVGDWRKNIDALSGMEGRKKKFES

SEQ ID NO: 2 - protein, 298 aa, Homo sapiens
MSDIEEVVEEYEEEEQEEAAVEEEEDWREDEDEQEEAAEEDAEAEAETEETRAEEDEEEEEAKEAEDGPMEESKPKPRSFMPNLVPPKIPDGERVDFDDIHRKRMEKDLNELQALIEAHFENRKKEEEELVSLKDRIERRRAERAEQQRIRNEREKERQNRLAEERARREEEENRRKAEDEARKKKALSNMMHFGGYIQKQAQTERKSGKRQTEREKKKKILAERRKVLAIDHLNEDQLREKAKELWQSIYNLEAEKFDLQEKFKQQKYEINVLRNRINDNQKVSKTRGKAKVTGRWK

SEQ ID NO: 3 - protein, 284 aa, Homo sapiens
MDAIKKKMQMLKLDKENALDRAEQAEADKKAAEDRSKQLEDELVSLQKKLKGTEDELDKYSEALKDAQEKLELAEKKATDAEADVASLNRRIQLVEEELDRAQERLATALQKLEEAEKAADESERGMKVIESRAQKDEEKMEIQEIQLKEAKHIAEDADRKYEEVARKLVIIESDLERAEERAELSEGKCAELEEELKTVTNNLKSLEAQAEKYSQKEDRYEEEIKVLSDKLKEAETRAEFAERSVTKLEKSIDDLEDELYAQKLKYKAISEELDHALNDMTSI

SEQ ID NO: 4 - protein, 1273 aa, Homo sapiens [sequence omitted]

SEQ ID NO: 5 - protein, 273 aa, Homo sapiens
MPEPGKKPVSAFSKKPRSVEVAAGSPAVFEAETERAGVKVRWQRGGSDISASNKYGLATEGTRHTLTVREVGPADQGSYAVIAGSSKVKFDLKVIEAEKAEPMLAPAPAPAEATGAPGEAPAPAAELGESAPSPKGSSSAALNGPTPGAPDDPIGLFVMRPQDGEVTVGGSITFSARVAGASLLKPPVVKWFKGKWVDLSSKVGQHLQLHDSYDRASKVYLFELHITDAQPAFTGSYRCEVSTKDKFDCSNFNLTVHEAMGTGDLDLLSAFRR

SEQ ID NO: 6 - protein, 22480 aa, Homo sapiens
  25bp deletion (21330)..(21356)
CTCAAATAATCTGCCTGCCTCGGCCTCCCAAAGTGCTGGGATTACAGATGAGCCACCACACCCGGCCTAACATTTTTAAAATTATAAACCCACTTCATACGTGTGACAATCCTGTGCAGTGGGTGCTGCTTTCACCCACCCCGCTTTACAGATGAGGAAACCAGGTGCTTTGAGATTAAAGAGATGGCAGTGCTAGGACTCAGAGACACCCTGGCCCCAGGGTCTGTGGACATGACCACTGCCTGAACTGCCTGCGAGGATGCTCCTGACCCGTTAGCCTTAGGGTTATGGGCCCAGCATGCTCCCACCGTACAATTTGCTGTAAGAGGGACACACACGGGGCTTGGAGTACCCCTGAGGCTTGCCAAAGGTTGGGGAGGCAGAATTGTGCTGCGGGGGGTGAGGGGGGAAGGGACAGGAGCCAGCCAGGGACAATGTGGCCCTACCTTCTCCCTGGAGAGACCTCAGCTCTCTGGAATTCATCTATATTTAGCAGGTGGCTGGACAGGAGGCAGATAAGCAGAGCCTGGGGAGGGGGGAGGTCCCCATATATAGTGGGAAGGACAGGACCCCACTCAGTCCCTCTTTGGGTGACCTGTGCCTGCTTCGTGCCTGGTGTGACGTCTCTCAGGATGCCTGAGCCGGGGAAGAAGCCAGGTAGCTTTAGGACTGGGGTTGGGTCTAAGTGTGGGAGCAGGGGGGTGTCTACAATTGGGGAGCAGGGCTAGGAGGGAGTTCTTGAGGGGGTTGACGAGGACGTGGCCTCTGAGCCCCAGGACAGGGGCAGCCAGTCCTCCAGGGTCCTTTTTTGATTCTCGGTTTCTTTTTCCCATCCCTCTGATGAGAGGTGAAGGTAAACCCAAGGTCAGGGACGCAGAGAGGGGACACAGCCAAGACAGGAAGCACCCGGGGTCAACCCATTCTGAGAATGCAAAGGGACCCCTTCCCTGTCCCATTGCCTTCACCTTCCACAGCAAGGTTCCCCGCGGACAGAGCCCACGTTTAGTATGGAGCTGAGCCAGGCTCACGGCATGCCTTCAGACCAGTCTCTACCCACTATGGGTCTCAGTCTCCCAACCTGGGGTGCCGGGGGTGGTGGTGGCATTGGGTTGGTTCCTCCTGGCTGTGACATTATCTGCTTCATGAGCCTGTACGGTGTGGCCCCCATGTTGAGGGTGACACAGCCCCTCTTCTCTGAGAGAGGACAGTCAGGAGCCCCTGAACCCAGGCCAAGGGCAGGGAAAATCACAGCCCAGGGACCTGGGCCTGGGCTGTGCCCTTATGAAGGTTGTCCAGGGCAGAGATCCAGGTCCCCATCCCCCAGCGCCCTCCCAGGCCAGCTTTATCTCAGTTGCTGATACCCAGCCTGAGGTCCTTGGAGGACAAATTTGGCCACATTCCAAGTAGGCAGACTTCCCCGCCCCCTGCCAAGGGCCCGAAATGGTTGGGGGAAAGGGAGCTGGGTTCATCACGGGTGTCTCCAAGCAGGGAAAGTGGACACATGCAGAAAAAGTCTTGTTGGGAGGTCAGGACCACAGCCCTGGCTCTCCCGACTGCTAGCTGTGTGATCTTGGAGGAGCCGCTTAGCTTCTCTGAGCCTCAGTGTCCTCCTCTGAGAAATGAACAGAATAACTGCAACTACCTCAGAGAACTGGTAGAGTATTACGTGAGGGGATGCATAGAAAGTGCTAGCACAGTATTTACTGAGAGGGGCTGCAGGGGCGGGGTGCACGCTCCAACCAGGGGCCGCTGTAGCCTCCACCTGGCCCTTCAGTCTCAGCTTTTAGCAAGAAGCCACGGTCAGTGGAAGTGGCCGCAGGCAGCCCTGCCGTGTTCGAGGCCGAGACAGAGCGGGCAGGAGTGAAGGTGCGCTGGCAGCGCGGAGGCAGTGACATCAGCGCCAGCAACAAGTACGGCCTGGCCACAGAGGGCACACGGCATACGCTGACAGTGCGGGAAGTGGGCCCTGCCGACCAGGGATCTTACGCAGTCATTGCTGGCTCCTCCAAGGTCAAGTTCGACCTCAAGGTCATAGAGGCAGGTAAGATCCTGATCAGCCTTCCCTGAGGTTTGGGCTGCTGGGGGAGGGGCAGCCCAGCGACTCTCCATCCATCCAGGGAACAGGAGGTGCTTTCACACTTTCTTGCCTTTGCTGTGGCTGTGCCCCCCGCCAGGAACGCCCTTTCCCCCTTTTCTGCACATGACCTCTCTGAGTACCCCCATGTGCCATCCTGGCTTGTTCCAGCCATGGGGTAGGTGGGAGAGATGAACAAGACACAACCCCACTCTCCCCCTGCAGACCTCCCTGGGGAGATGACCCTGCACAGACGGCCGGGAAGCCCCAGGAGCTGTGGATGAGGGGAGTGCAGCCCAGTCTGAGGGGCTGCTCTAAGGCTGGGGCAGAGCAGGGATGGACCTGGAGCTGTTCAGGGCAGATGCAGAGGACATGGGAACTGATGGCCTGGGACGGGGAGGAGAATGTGAGAGGCAGGCTGGCTCTCCAGCTCAGCATGAGCTGCCTCTGAGGGGCCCAGGCAGCGGGAGGACAGCCATGGCAGACTTTCCTCATCCACAGCGGGCTCATGGGTCCAATGCTCAGGAGAGAGAGGGTGGAGGGGTCTCTGGTTAGTGGCTGCACCACGACTCCCATCTGATCCCCTCCAGAGAAGGCAGAGCCCATGCTGGCCCCTGCCCCTGCCCCTGCTGAGGCCACTGGAGCCCCTGGAGAAGCCCCGGCCCCAGCCGCTGAGCTGGGAGAAAGTGCCCCAAGTCCCAAAGGTGAGAGGGGCTGGGCAGGGAGGGGCAGGATCCGTGGGTAGGGCGTGTCCAGGGCAGGTCTCAAAAGCCTTTGCTGGGCCAGGTGCGGTGGCTCAGGCCTATAATCCCCAGTACTTTGGGAGGCCGAGGTGGGCGGATCATGAGGTCAGGAGATTGAGACCATCCTGGCTAACATGATGAAACCCCATCTCTACTAAAAATACAAAAAAATTAGCCGGGCGTGGTGGCGGGCACCTGCGGTCCCAGCTAACTTGGGAGGCTGAGGCAGGAGAATGGTGTGAACCTGGGAGGCGGAGCTTGCAGTGAGCCGAGATTGTGCCTCTGCACTCCAGCCTGGGTGACAGAGCAAGACTCCATCTCAAACAAACAGAAAAAGCCTTTGCTCACAGGGTCAAGCTCAGCAGCTCTCAATGGTCCTACCCCTGGAGCCCCCGATGACCCCATTGGCCTCTTCGTGATGCGGCCACAGGATGGCGAGGTGACCGTGGGTGAGTGTGAGCTGCTGTGCCCAGCATTGGGGTGGGAAGGGGGGGCAGCAGGACACTCCCCAAGCCGGGCCTGTCGCCCCTGCCTTTGCAGGTGGCAGCATCACCTTCTCAGCCCGCGTGGCCGGCGCCAGCCTCCTGAAGCCGCCTGTGGTCAAGTGGTTCAAGGGCAAATGGGTGGACCTGAGCAGCAAGGTGGGCCAGCACCTGCAGCTGCACGACAGCTACGACCGCGCCAGCAAGGTGGGTCCACCGGGGTCGTGGAGACACGGAGAGAGGGGACATGGAGAGCCCTAGAAGGCACAAGGCACACAGAGGGCACCATGAGACCCAGAGGCACACACGAGCTGCAAGCAAGGGGTGTGGGGAATCTGGGAGGCAGGTGGAGGGGCCAGGGGCCCACAGGGAGATCCCGGAGGCCTGGGGGCCAGGGAGTGCGATGAGGCTCATGGGAGCACTGTCGGGGACCTGGGAGACTTGGTGGACATGATCACCTCAAGGGGGCACCTAATAAGGTTTGGCCGGGGGTGGGGGGTCGCCCAGGGTGGTCAGGAAGCCTGGAGGACACTGTGAAGCATGGGGGCACCAGCTAGTGGGGTTGTAAAGCCCCTGGATGGGTGAGTATCAGGGCTAAGGGCTCAAGGGACTCTCAGGGATTAACCAGAACAATTCCTCATTATACAGACACGGAAACCAAAGCTTGAGAGGTTAAGTGGCTGCTCCAAGGTCACTTAGACATAGGCCTAGAACCTTAAGCCTTCGTTTTTTGTGTTTTGTTTTTTTCCGCCCTGCCCACCAGAACCTTAAGGCTTAAGTCCCCTTGTTTGTTCACTCCAAGCCTCTGCTTTTCCCAACCTTCCCCAGTCCTGGGCTAGGCAAGGGGGCCTCTAGCAAGAGAGGCCAACATGTTCTGCTGTGCGGGGCTGTGCATTGCACAAAAGCCTGTATTTCACCACAACCATGTGCCAGCAGATGGCAATCATGGGTTTCGAGATAGTCCCCTTTCTCTGTACAAAAACTCTGTATGGGTTAACTAAAGCCTGAGGGTGCGAAGATCGATGAGCATTTGTCACTCCCAGCCTCCTTTAAATATATATATATATATATAATTTGAGACAGGGTCTCGCTCTGTCGCTCAGGCTGGAGTGCAGTGGCTCAATCTCGGCTCACTGCAACCTCCACCTCCTGGGTTCAAGCGATTCTCCCACCTCAGCCTCCCGAGTAGCTGAAACTAGAGGTGTGCACCACCATACTTGGCTAATTTTCGTTATTTTTAGTAGAGACGGAATTTCACCACGTTGGCCAGGCTGGTCTGGAGCTCCTGGTCTTATGTGATCCGCCCGCCTCAGCCTCCCAAAGTGGGGATTACAGGCCTGAGCCACCGCGCCCGGCCACTCCCAGTCTCCTTTAAGGGTGCGGAGCCTTGTCTCCCGGCCCCTGGTGTCCCCTGACGCCCCGTCCCTCCATGCACACAGGTCTATCTGTTCGAGCTGCACATCACCGATGCCCAGCCTGCCTTCACTGGCAGCTACCGCTGTGAGGTGTCCACCAAGGACAAATTTGACTGCTCCAACTTCAATCTCACTGTCCACGGTGAGGGGGCCCTGGTGTCTGTCCTGGGCTCGGGCTCCCCATGGGTCCTGGTCTCCTACCTCCTTTTCCCAACACTAAGGAGGATGCCTCGTCCCATCCAGACATGAGTGCTGGCCACGTGCCCAGTGCTGCACACACAGGGTGTGAGAGAAACCCCAAGGCTTGCAGGGTAGGCGTGGGGGCTTAGGGCTGAGTCCGGGTCTCATCTGGGTGGGACTCTGTTTTATCATCTTGGTGTCACGGCTCCTGGCCCATGGCCTGGCACTGTCTGAATGTTTGGGCGATGGGTGAAGGTGAGTTGAGAGATGGGTGGGAATTGGGGACAATAAACCGTCCCACCTGCCTGGTACCTTGACCCTGGGAAAGGCTGGGAAGGTGAGATCCTGGGGCAGATGCCCAGCCTCATGGGGTCATGAATGGGCAAGTCTGTGAATACTCAGAGGCCGCCCCCAGGGCAGGGCTTCTCAAACGGCCCCCTCTGAAGCCCCTTCCCCCATCTCTCCACCCTTTGAACCCAGAGGCCATGGGCACCGGAGACCTGGACCTCCTATCAGCCTTCCGCCGCACGTGAGTGGCCATCCTCAGGGCCTGGGGGAGGCCAGTGCTGGAGTCTGAGGCCCTTCAGGGTCTCGACTGGGGGTCAGGGCTGGGGATGATTTGCGGGGCGGAGCTGAAGTCAGTGGGGGCTGCAGGGGAGAGCAAGCTTCCCAGGCAGGTGAGGATACTGAGTCTAACCCCCACAGGAGCCTGGCTGGAGGTGGTCGGCGGATCAGGTACCCCTGCCCCAGGCCCTACCTGCACCATCCGCAGACCCCCAGGGGTCTGAGACGGGTGGGAGCCCAATCTGGCTAGTGTCCCTTTCTCCCTCCCCACTCCACTCCCATCCTGCTCCTAATCCCTTTCCAGTCCCTCACTGCAGCCTCACTGGGGGTCCCTCTCCATAGTGATAGCCATGAGGACACTGGGATTCTGGACTTCAGCTCACTGCTGAAAAAGAGGTGAGTCCTGGGTGGCAGTTCCTGGGGCAGCTGGTGAGGCCTGGCTCCAAATCCCAGCACTGTGGTTTGCCAGCTGTGTGACCCTGAGCACAACACTGCACCTCTCTGAGTTGGTTTCCTTGTCTGTCGAGGGGATGATCCCAGTGTGGAAGGAGTTAAAGGGCTCTGCAGACATTATGTCCCGTCAACAGTCATCCTCACAGTGTCCCCCACGGCCACTCCACACTTGAGGCTGCAAAAGCTCCCTCCGTAAACTCCGGGGACCCAGAAGGAACAGAGAGAGGGTCCCCAGAGCTGCAGGGTCTACCAGGTCGGCCCAACTGACTTATGTTCTCTCTGCCCTCTCTCTCCCTCTCCCCCGGCCCCTCCATTATGGCTGCTGCTGCTGTGGCCCAGAGAGTAAGAATCGGGGTCTGGGTGTTTGGGGGCGGCAGGGCGCTGGTGGGTGCAGAGGGGATTCCCGTCCCCTCTCTGAGAGGCTGTGGTGAGAGACTCAGAGCAGGGTGCGGCTCCCCACGGACAGGGGTGAGTCTCTGTGCCTTGGGCTTCTCCTGCCACCCACCTATCTGTCAGAGCTTAGGAGGGATAAGAAAAAGTAATGCACACAGGAGGTCCTGCCAGAGCCTCCGGAGCAGCATGGGTGCCCCACAACACAGGGAGTGCAAGTGCGGAAAATAGGGAGGAAGTAAAGGCCCATGGGTGGGGTCCAGGTCTTTGAGGGAAGGTGGCCATACCTCTCATGTGCCACCTACCCTTTCTCCCACCCCCACCCCTGGAGCAGCAGTTTCCGGACCCCGAGGTGAGTGCCCAACATAGCACAGCACCCTCATCACCCCTAATTCTGCCAGGAGCCCCTCCAGATCCCCAGCCCCTCTTCAGCTCCCTTGGCCCCCAGTCCTGCCTGGCCTGGGACCCAGAGCAGCTCCAGCTGCCCCACCAGAAGGGAAGGGGCAGAGGGACAGGGGTGGCTACAGCTCCTTGGTCCTGGGCCCAGGAAGCCCCGCCCAGGGGGCTGCAGTCTTGCCCCCGGCCACAGCCTAGACTGCGGGACACAGGGACTCGAAGCTGGAGGCACCAGCAGAGGAGGACGTGTGGGAGATCCTACGGCAGGCACCCCCATCTGAGTACGAGCGCATCGCCTTCCAGTACGGCGTCACTGACCTGCGCGGCATGCTAAAGAGGCTCAAGGGCATGAGGCGCGATGAGAAGAAGAGCACAGGTTAGCCCTTCCTCAGAGGGGAGAGGAGAGGGCACAGGCTAGCCCTTCCCTACACAGGAGAGGAGAGGGCATAGGTTAGCCCTTCCCCACAGGGGAGAGGAGAGGGCACAGGTTAGCCCCCCACCTCGTCCACATACATGGAAAGAGCGGAGTCCGGCTTAGCACAGAGACTCACAGGAAAGAAGAGCCCAGGTTAACCCCTCCTTGTGTAGAAAAAGCCAAGAATGCCCAGGTTAGCCCCTTACCACAGAGATAGAAGAGAAGAGCAAAAGTTAGGCCAGGACAGCCACAAGGAAAAGGACAGAGCTCAGGTTAACCCTCCTCACCAAGGAAAAATCAGGCTACACTTTCTTTCTCTACCCTCTTCTGAAAAGAAATGAAGCCCGGTTAACCCTCTCCACACCCAAAAGAAAAGGGAAGAGGCCCGCTAAGCCTGGAGAGCCACACACAGGCAAAGAAAAGAGCCTGGCTAATCTTTCTCTCAGGCAAACAAAGCAGGAGGGCTTGGGTTAACTACCCTCACCCCCAGGCAGATGGAAAGAGGAGAGCCCAGGAATAGCCCTTCTCCACTGAGGGGTTGACAAGGGCGGGTGTGAGCCGGGAAAGCAGGGGTCTCACATGCCTTCTCCCCACCATGTCTGGGTCTAGCACAGGGCCTGGCACACACCTCGAGCGCTCTATAAACACCGACTGAATGGAGGGGTCAGCTTCCATGCAGGATATGGGAGACAAGCCCCTCCCAAAGCACACACACAAAACACACACGATCATGGGGACGGGGAGAGGAGCTGGTTAGTCCCTCCCTGCTGACCTCCCCCAGGCCCCTTATCCCTCTGCCCTGCCTCCCTGTTGTCTCAGCCTCACTGGGTTTCCCATCACCCCATCGCTAGGTGGCCTGGCCCTTACCGGCACCGTGGCCCCATTTAAGCCAGGGATCCCCTGAATGCCACCAATGTTCCTCTGGGTTAACTCCTGAACTGCTGAGCCCAGAGGGCACCAAGCAAAGGACTGTAGGTGCCCAAGGAGGAAAAGATGGAGGCTGGCAAGGCAGATGGGGACCTCCGAGGGTAGGGGTGTCTTCCAGGAATAGCAACAACAGTAAAAAGAGCAAATTGCTGGCCAGGTGCAGTGGCTCACGCCTGTAATCCCAGCACTTTGGGAGGCCGAGGCAGGTGGATCACCTGAGGTTAGGCGTTCAAGACCAGCCTGACCAACATGGTGAAACCCCGTCTCTACTAAAAATACAAAATTACCTGGGCATGGTGGCAGGCGCCTGTAATCCCAGCTACTCCGGAGGCTGAGGCAGGAGAATCACTTGAACCCGGGAGGCAGAGGTTGCAGTGAGTCAGGATTGTGCCATTGCACTCCAACCTGGGTGACAGAATGAGACTCCATCTCAAAAAAAGTGAGTGAGTTCCTTCCTAAGCCAGCCACTAGGTGCTTTATGTAATTATCTCATTTAATTCTCGAAACAACACTATGATGGAAGGACTATTACCATTTCCATGTTACTGAGGAAACCGAGGCACAGAGGGTTTAAGGAACTAGCGCAAGGTCACAGAGCACCCAGCAGTAGATCTCTGCCCCTCCGCAGCTGACTCCAGGCCCAGCCAGAGTATTTGAGGACTCAGGGGTCCTTCTCTGCTCGGTGGATGGAGCCCCCACTTCCTCCCTGGGCTCAGCAGCAGCAACATGGGCTTTTACAAACACGGCCGCAAGAGGAGGCATTGTTGTGGAGCAGGGCTGGGCTCAAGACCCAGCTCTGCCACACACCTGCTGGGCTGGTGGCATTGGGCCAGTCGCCTCTCTGAGCCCTATTTCCCCCACTGCAGCAGCATGGGGACCACCGCACTGGCCCTGGGGGCTGAGATGGGAGTGAGAGTGCTGGGCACAGAGGGCTGCTAGGTGGGAGCGCCTGGGAAGAGTACACAACTCAGGGGTTGCTGAGAGAAGAGAAGGCACAGCTGTCCCAGCCCCAGTTCTCCTGACAAGCTTCTGTCTTTCTGGAAGCCCCTAACCCAATCCCTGAGCTGAGCAGGTGGAGAGGACCCCAAGGAAGACAGAAGGACAAGGTCAGACAGACAGAGACAAGGAGGTGGGTTGACACATAGACACAGATAAAGAAATGAAGACACCATGCCTGTAATCCCAGCACTTTGGGAGGCTGAGGCGGGCGCATCACCTGAGGTCAGGATTTCAAGACCAGCCCGGCCAACATGGTAAAACCCCGTCTCTACTAAAAATACAAAAATTAGCTGGGCGTGGTGGCACATGCCTGTAATCCCAGCTACTCGGGAGGCCGAGGCAGGAGAATCACTTGAACCTGGGAGGCAGAGGTTGCAGTGAGCCAAGATGGCGCCATTGCACTCCAGCCTGGCCAACAGAGCAAGACTCTGTCCAAAAAACAAACAAACAGACAAACAAACAAAACAAAAAGAAATAGAGACACAGAGAAGTGGAAGCGGGGCGGCACAGAGGGGATTGGAGGGGGGGACCTGGGCCCCAGCCACAGCCACAGTAGCTTGGCCTGGGGGAGCAGGGTGCGGGCGGTGGGGTGGCCGGGGCTGAGGGGTGGTGCTCAGCCTTTCAGAAGAAGCTGGAGCCGGCCTACCAGGTGAGCAAAGGCCACAAGATCCGGCTGACCGTGGAACTGGCTGACCATGACGCTGAGGTCAAATGGCTCAAGAATGGCCAGGAGATCCAGATGAGCGGCAGGTGCAGCCTGGGGTGGGGAGGGGGGCTCGGGTGGAGGTGGGGACCCCCATAGCCTTGCCTCCTGCCCCTCTCCACATGCGTATCTCTGACTCGGTGTGGCTCTCAGCCCCATCTCTCTGGGCCTAATTTCCCATCCTTTTGCTCCTGCCGGTCCCTCTCTCTCTCTCCTTTTGTCTCGGGCTCACTTCCTCCCGTCGGGAACACTTCAACGGCCCCTTCTGTTCTACAGCAAGTAAGTTCCCCTCTGGATGGCTTGGGGTGGGGGGCACAGGGATTATCATGGGGCGCACTGGGCCCGGAGGGGTGTCCGCAGCTTTCCTGCCACTTCCCTGCGGCCCCCACCCAGGTACATCTTTGAGTCCATCGGTGCCAAGCGTACCCTGACCATCAGCCAGTGCTCATTGGCGGACGACGCAGCCTACCAGTGCGTGGTGGGTGGCGAGAAGTGTAGCACGGAGCTCTTTGTGAAAGGTGGGCCTGGGACCTGAGGATGTGGGAACCTGGGGAGGAGATGGCCTCAGGGGAGCCAACCCTCATGCTCACCCTGCCTGGACAGAGCCCCCTGTGCTCATCACGCGCCCCTTGGAGGACCAGCTGGTGATGGTGGGGCAGCGGGTGGAGTTTGAGTGTGAAGTATCGGAGGAGGGGGCGCAAGTCAAATGGTGAGTTCCAGAAGCACGGGGCATGGGTGTTGGGGGCATCTGCCCAGAAGAGGCCACAGCACTTGCCACCCACCCACCCGGCTAGGCTGAAGGACGGGGTGGAGCTGACCCGGGAGGAGACCTTCAAATACCGGTTCAAGAAGGACGGGCAGAGACACCACCTGATCATCAACGAGGCCATGCTGGAGGACGCGGGGCACTATGCACTGTGCACTAGCGGGGGCCAGGCGCTGGCTGAGCTCATTGTGCAGGGTGAGCCTGGCTGGGGGGGCACATGAGGCTTTAGGGCTTGGACCCCTCAGCCCCCACCCCACCTAGCCCTGTAGGGGAGCAGGCAAACCTGGGCTCAAGGCCTCTGTGACCTTGGGCCTTTGACAGCCTAAACCTCATCCTTCTCATCTCTAAAATGACGATGCTGAGGTGACCGCTCCAGAGGGTACTGAGAGGGTCATACGGGCCTGGTGAGCATCACCACCCCTCAGACACTTGAGGTTCCTTATGTGCACTGCACCATCACAGGCAAACATGGCACACACAGGCACACGTGTTTTCACATGCCCACATGCACCCAGACACGTGCGCACCAGCGCCCATGGGCCCACACGCCCTCCACAGGGATTCACGCCACACCCACACACCCTCCCGAGCTCAATGGCTCTGCCCTGCCCTGCAGAAAAGAAGCTGGAGGTGTACCAGAGCATCGCAGACCTGATGGTGGGCGCAAAGGACCAGGCGGTGTTCAAATGTGAGGTCTCAGATGAGAATGTTCGGGGTGTGTGGCTGAAGAATGGGAAGGAGCTGGTGCCCGACAGCCGCATAAAGGTGTCCCACATCGGGCGGTGAGTGTGCAGGGCAGGTGGATGGGACAGGTGGAGACTGAGATGGAGACAGAGAGAGACACAGGGAGAAACAGAGAGAGAGAGATAAAGACAGGTGGACAGAGAGGCAGATGCACAGAGACAGGACAGAGACTGAGACAGAGACCCACAGAGAGGGAGAAACAGACAGACCCAAAAAGACAAAGAGACAGAAAGACAGAAATAGTTCTATAGAGACAGAAAGATATGCACAGGGAGGCACACACATAGGAGAGTTCGTCAGAGACAGAGAAGAACAGAGGGACAGAGACAAAGAGGCAGCAGGGAGAGACACGGACGGGGTCGAGCGCTCCCCAGCACCCTCCCCCAGCTTGTGACCAGGGCAGGGCAGGAGGCCACCAAGGAGGCCGGCTCTGGCCAGAGGCCCTCACTGCCTCTCCCGCCCTCTTGCTCATCCCTGGAGCAACTGGGCTGGCCGTGCTGGGCCCAAGTTCACAGAGATTAATGAGACCCAGCCCCTGCCCAGTCTCCAAGGGCCCAGCCAAGGAGATGCTTTGATGTAGAAATCACTTTTACGCTGCCTACCTCTGAAGGCACATTAAAAAGCTGGAAAATTAGATAATATAGAAAAAAAAAGGCATTTTTAAAAATCAGAATACCAACAAGCCAGGACAAGGTGAGGGCCTCCTGGGACCCTGGCTGGGGTATCTGGCAAGGCCAGGGGTGTGTGGCCCAGTGGGGTCCCCTGAGCCACTGCTCCCCTGCAGGGTCCACAAACTGACCATTGACGACGTCACACCTGCCGACGAGGCTGACTACAGCTTTGTGCCCGAGGGCTTCGCCTGCAACCTGTCAGCCAAGCTCCACTTCATGGGTGAGCCTGCTCCAGGGTAGGGTGGGTCGGGGCAGTGGGGCCAGGAGCCCCTGTCACTGGGCCCTGCCTTTGCCCCCGTGCTACTTGCTCTTCCTTCTCTTGCAGAGGTCAAGATTGACTTCGTACCCAGGCAGGGTAAGTCTTGGGGCCCCTGAGTCTTGGTTCTGCTCACTTTCCCGCAACCCACCCACGCAGGCCTCCCCTCCACTCACAGAGGGAAGAGCAAGGATCAAAAGTGGGGGTCCTGGGGCCCAGCTTCAGCTTTGCCACTCATGACCATGGGTCAATTATAATCTCTGTGAGCCTCAGTTTCCTCTTCTCTAAAATGGGAACCCTGATAGTGCCACCTCATAGGATAGTTATAAAGATGAGGTGACATGATGTATCAGACCTGTCAAGTGCTGTCACATGTGTGACTGTTATTATTACATCTTTTCTTCTTCTTCCTTTTTTTTTATTTTTATTTTTTTTGAGACGGAGTCTCCCTCTGTCGCCCACGCTGGAGAGAAGTGGCGCGATCTCGGCTCACTGCAAGCTCCGCCTCCCAGGTTCACACCACTCTCCTGCCTCAGCCTCCTGAGTAGCTGGGACTACAGGCGCCCGCCACCATGCCCAGCTAATTATTTTTTGTATTTTTAGTAGAGACGGGGTTTCACCATGTTAGCCAGGATGGTTTCGATCTCCTGATCTCATGATCTGCCTGCCTCGGCCTCCCAAAGTGCTGGGATTACAGGCGTGAGCCACTGCGTCCAGACTGCTTTGCTTTGTTTAACTGAGGGCAGATTCCTGATTTATTTGGCCAGGGAACCTTTTTAATGTGTGCATGTGTGCGTGCATGTGTGCGCGCGTGTGTGTGTGTGCATGTGTCTGTGTGTGTATGTATATATGTGTGTCTGTGTCTGTGTGTGTGCGTGTATGTATGTGTATTTGTGTCTGTGTGCATGTGTGTGTCTGTGTGTGCATGTGTGTATGTGTATGTGTGTCTGTGTGTATGTGTATATGCGTGTGTGTGTGTGTATTGTGTGTGTGTGTATGTGTGTGGCAAGACACTGATGTTCCCCCAGTTTTCAAAATGCTATGCTAGGTGACCTGACCTGAATATTACAAGCCTCCCAGCCAACGTCCTGGGGTGATGGGAATGTTCCAGATCTTGGTTGGATTAATAATTGCATGGGTGTATTCATCTGTCAGAACTCAGCCTACTGTCAGATCTGAGCATAAACTATACCTCAATACAAATCTAAAAAACCAATAACCAGGGGCCCAGGCTCTTGAGCAGCCCCGCCCCAGTGACCTGTGCTCCTCCTGGCTCTCCCGTTTCTCTGAACTACATTGTGTCTTCTGCAGAACCTCCCAAGATCCACCTGGACTGCCCAGGCCGCATACCAGACACCATTGTGGTTGTAGCTGGAAATAAGCTACGTCTGGACGTCCCTATCTCTGGGGACCCTGCTCCCACTGTGATCTGGCAGAAGGCTATCACGCAGGTACTGTGGGTCCCTCCTCAGTCTCCCCATCTATAAGATGGGTGTGTGGAACCAGCCAAGTGCTAGGACCTGCTGCACACCCCACCCACATGTGCACGCCCTGCTGTTGGCTACCCCATGGGAGAGACTGGTCAGGGGGCAGGCAAGGTGGGCAGTGTGGGTCAGGGGGTGGAAGAAGCAGCAGAGGGGCGCTTCCAGGCTGAGTCAGCTCCTCTGCTCCCTACTTCCCTCCTGCCCTGTTCCCAGGGGAATAAGGCCCCAGCCAGGCCAGCCCCAGATGCCCCAGAGGACACAGGTGACAGCGATGAGTGGGTGTTTGACAAGAAGGTGAGTGAGACTGAGGTCAGGAGGAGGCTCGTTTGTCTTTCATCACTTTCTACCTACGTGGTGCCGAGCCCTTGGCGAGGTCTCCATGGAGGTGCCACACTTGGCCGGGTGTGTTGCTGCCCCCAGCAGGGTCCCTGTGGGGCCCTCACACCTCAGGCAGAGGGTCTCTGTGAAGTGTGCTGATGGCCATCACAAGGATGTGGACACCCTCTCATTGCCAGGAAGCCCTCATGACTGGGCTGCTCCCTGGCTCCCTTCATCCTAAGCTGACCTTGACCTTGACCTTGTGCTCCCTCTCAACTCAGGAGTAGTTTCTGAGACACTTTTCTGCCAGTGTAGCTGCCGGGGTCTTGCTCCTGCCTGGCACTGTTGGTTCTTCTGAAGGCTGGGGTCTTGAGGGGTCCTCATGATAGCTCATCTGAGAGGACAGGTGGTTGCCCCAGGTCCCGTGACAAAGCTAGAACCCGAGCCCCTGCCCTCAGTCGGTGCCACAGAGATGATTTTGAACTAGATGCTGACGTGGATGCAGTCTTCCCACAGGCTGTGTTCCCCCCATGGACCCCCGGGACATCTGGCTGTTGGCGGAGGCTGCAAGGGCCTCTGGGGTCTGACTTGGATCTCACCCCAACTCTGCACCCCCCCAGCTGCTGTGTGAGACCGAGGGCCGGGTCCGCGTGGAGACCACCAAGGACCGCAGCATCTTCACGGTCGAGGGGGCAGAGAAGGAAGATGAGGGCGTCTACACGGTCACAGTGAAGAACCCTGTGGGCGAGGACCAGGTCAACCTCACAGTCAAGGTCATCGGTGAGGCCGGCCGGGGTCCAAGCTGGAGAACACAGAGGGGCAGCCCCAAGGAGGGCCCATCCGTTCGCTCATTCAGCCACTCGACAAACATCACGGGCATGCTCCCTGAGCCGAGCGGCAACAGGACAGGTTTTTCTGCCCAAAAGGAGCCCCGCTCTGGGGGAGATGGAGGCAGATGCATCCAGCAGATTTCCTGCGACACTGCTGTACATGAGCCATGTCCAGAGTGCCGTGGGAGCACTGAGCAGAGAGCCATCCCTGTGCCTGGGGGTGATGCCTTTAGGAGGCTGAGAAGATGGTGAGGAAAGGCATCCCAGGCAGAGGAAACTGTGTACAAAGGGGTGGAGGAGGGAAAGGGCCTTTTGGGTGTGGAGAATGAATGAAACATGTCAGGAGAGGACTGGGTGGGGTTGGGAGGATCAGAGCCAGGACAGGGGTGAATATGGGCAGTGAGAAGCCTTAAAGGGTTGACGGAGGGCTGGCGACATGATCAGAAATGCATCCTGTTGAGACCCAGCCTGGACAACACAGTGAGACTCCATATCACTAAAAAAAAAAAAAAAAAAAGGAAAAGAAGAAAGAAATGCATTCTAGAAAGATCTCTTTGGATCTGGGGGTGGTGGGGGCAGCCTGTGGCGGTTAGTTGGAGTGGGAAGGGGACGAGCAACGTTACTCAAGGCCCTGAGCGGGGCAGGGCTGATGTGGGTCCATCCCACCCCATCCAGACGTGCCAGACGCACCTGCGGCCCCCAAGATCAGCAACGTGGGAGAGGACTCCTGCACAGTACAGTGGGAGCCGCCTGCCTACGATGGCGGGCAGCCCATCCTGGGTGAGTGCAAGGGCACCGGATGGAGGTGTGAGGGCGCCAAACAGATCCGAGGGAAGGTGGTGTGGGGATGCCTGGGTTCCAGACCAGAGCTGCCACCTCCCCTGAGCCAGGCTACATCCTGGAGCGCAAGAAGAAGAAGAGCTACCGGTGGATGCGGCTGAACTTCGACCTGATTCAGGAGCTGAGTCATGAAGCGCGGCGCATGATCGAGGGCGTGGTGTACGAGATGCGCGTCTACGCGGTCAACGCCATCGGCATGTCCAGGCCCAGCCCTGCCTCCCAGCCCTTCATGCCTATCGGTGAGCCTGCCTGGCCTGGTCCTGCCCCCCGCCCCCTCCCCAGTTAAAAACCTCCAATAATAGCAGGTGCTCTGCAGGCAGCCATGCTAGACACTCACATCCACAGTCTCCTTTCATCCTCGACTGGGGATACTCAGCCGCATTTTACAGATGAGGAAACAGGCTCCAAGCAGTTAAATGACCTGCTTGAGATCTCCCAGCTGGTTGGGGTGGAGCTGACTCTCACACTACGCCGACCCCTAGTTCACGTTTCAAGCCTCTCTTTCCATGTCTACAAAATGGGAATAATGACACTGCCTATCTCAGAGAGCTGTTAGGATGATATGGGATGACCAGGCAGGCCATCTGGCATCTAACAGGTGCTCAATAAATGTTCATTGTCCCCTGTTCTCCACACCCTTCAACCCAGGGTTTTCACCTATGGGATGGGGCAAGGGCTGTAGTTAGTGGAGGGGTCTCAGGGGCATCTGTCTGTGGAAAGAGTCCTGGACCAAGACTCAGAGATGGCAGAGGCAGCTCCATGGTCCCCGGGGGGCCAACTGGAGTCATCCCTCCCTGAAGCCTCAGCTTCCTCATCTGTATAATGGGGTCTGAGTGGGCAGAACAGGTGTTGAGACTGTGGCAGGGCTCAGGAGGACACCTGTTCCCACTAGGGTGGGTGAGTCCGGGGGCAGCTGCCTGTGGCAAGGGCAGGCCAAGGACCCCTGGGCCCAGGGATGGGGCACAGCTTGGGAAGATGGCAGGAATCGGGGCCTGTGGGGAGAAGGGTCCAGGCCTCTCGTTTTAGGGACTGATTCCTGGTTTGTTCAGTTCTTAGTCAACTCACCTAATCATACGTGCCAGGAAATTCCCATGTTTTCTGTAGTTTTAGTTTTAGTTTTTTTTTTTTTTTTTTTTTTTTGAGATGGAGTCTCGCTCTGTCACCCAGACTGGAGTGCAGTCCTGGCGCGATCTGGGGTCACTGTAACCTTCACCTCCCAGGTTCAAGCAATTCTCCCACCTCAGCCTTCTGAGTAGCTGGGATTACAGGCATCCACCACCATGCCCAGCTAATTTTTGTATTTTTGTAGAGACGGAGTTTCACCATGTTGGCCAGGCTGGTCTTGAACTCCTGACCTCAGGTGATCCTCCCACCTCGGCCTCCCAAAGTGCTGGGATTACAGGCGTGAGCCACCGTGCCTGGCTGCTGTTGCTGTTTGTTTTTAAGCCAATCTACAGACATGTCTTGTAACAGGGACAAAATATTCCTTAAAGTGGCCAAAAGCCAGCTGAACAGGAAGTGCCCCCTATGTGACCAGTGGGCAGTTCAGAGTCTAGGGCATGGATCTCCAGCTTCCCCAGGCTTGCTCAGACCCCTCTCTGACCTCTCCTCTGCCCAGGTCCCCCCAGCGAACCCACCCACCTGGCAGTAGAGGACGTCTCTGACACCACGGTCTCCCTCAAGTGGCGGCCCCCAGAGCGCGTGGGAGCAGGAGGCCTGGATGGCTACAGCGTGGAGTACTGCCCAGAGGGCTGTGAGTGTCCCCGCCCCCAACCCCCCTCCCCAAAGGAAGAACATGCTCACCTTGCCATTGAGCAGATTCACCTGTAGTCAAGTTTTTTAGGCCCACTTTTGCCGAAAGTTGAGGACACCCAGAGAAATATCTGTCCTGTTTTCAGAAGTAAAAAAGCTTGCCTAGTGAAAAACAAATGCAGAGTAAAGCTGACTGTAACACTTCTTTGAGCAGTGCGAAATCAGCAACTACATTTTAAAAACATATTTAAGGCCAGGCGCGATGGCTTACGCCTGTAATCCCAGCACTTTGGGAGGCCAAGGTGGGCGGATCACCTGAGGTCGGGAGTTCGAGACCAGTCTGACCAACGTGGAGAAACCCCGTCTCTACTAAAAATACAAAATTAGCCGGGTGTGGTGGTGGGCACCTGTGATCCCAGCTACGCGGGAGGCTGAGGCAGGAGAATCACTTGAACTCGGGAGGCAGAAGTTGCGGTGAGCCAAGATCATGCCATTGCACTCCAGCCTGGGAGACAAGAGCGAAACTCCATCTCAAAAATAAATAAACAAATAAAAATTTAAAAAAACATATTTAAATCCTGGAGATTCCTATCAGAGGAGTGGGCAGTGGGAGTGGGGTGTCAGTGGTGACACAGCCTGTGGCCTTGCCTCCCCCTCCCCACCCCCAGGCTCAGAGTGGGTGGCTGCCCTGCAGGGGCTGACAGAGCACACATCGATACTGGTGAAGGACCTGCCCACGGGGGCCCGGCTGCTTTTCCGAGTGCGGGCACACAATATGGCAGGGCCTGGAGCCCCTGTTACCACCACGGAGCCGGTGACAGTGCAGGAGATCCTGCGTGAGTGCCCCTTTGTGCAGTCACAAGACCCGCCATTGACACCCCAGGCCCTTGGTGTCCAGTGGAGGTGGGGACAGTTGGGGAGGCCCAGAATGCTCTGCCCAGAACGCTGGGCAGAGACAGGAGGGGTTGGAGCCATCGTCCATGCGCTAGGCCTAGGGAAAAAGTGCAGAGAGGGTGGCCAGGCACAGTGGCTCAAGCCTGTAATCCCAGCACTTTAGGAGGCCAAGGCAGGCAGATCACTTGAGGTCAGGAGTTTGAGACCAGCCTGGCCAACATAGTGAAACCCTGTCTCTACTAAAAATACAAAAACTAGCCAGGTGTGGTGGCGCGTGCCTGTAGTCCCTCAACTACTTGGGAGGCTGAGGCAGAAGAATCGCTTGGACCCGGGAGGTGGCAGTTGCAATGAGCTGAGATCACACTATTGCACTCCAGTCTGGGTGACAGAGTGAGACAACGTCTCAAAAAAAAAAAAAGTGCAGAAAGGGCTCTCTTGGTGTATGTCAGTCCCTTAGTTCTGGAACAGGGAAAGTAAAAGCCACAGTACCCAAGCTCTCTTCCAGGAGTGTCTCAGCTCCAACAGGTAAATATTGTCTATGTAGTCAGATTGGTCCCTCTGGGATCCACTTCCCATTCCTTTGTTCCTTGTCCCAACAAAGGGTCCATCTTTTGTAAGGATTGGTTCTCAAAGCTGGAACGAGGCAGCAAACTCAGGTGGTAACAACACAGGGCATAGGGTGTCAAAGCCCAGGCTTACACCCTGGTCTATGACTTACTAGCTGTGTGACTTCAGTCAAGTTGCTTACCCTCTCTGATCCCTGCCTTTTGAAGTGGTGAGGCTCGGGGAAGCCAGCTGGGCTTGGGCAGCCTGGAGTTGCTGTGTTAGCAGGAGCTAAAGGAGGCGCAGGGCCCAGCAGGCAGCTTTTTTCTTAGCTGCAGCTCTCTGGGCCTTGTCTCAAGGGAGGTTGGAGCTGTGGAGCCCCTCAGAGCTGTGGCGGGCCCTCACTTAGCTACCCACTCTATACCCACAGAACGGCCACGGCTTCAGCTGCCCAGGCACCTGCGCCAGACCATTCAGAAGAAGGTCGGGGAGCCTGTGAACCTTCTCATCCCTTTCCAGGTGGGACTGGCCCCCTTCCCTGTCCCCCAGGGGAGAGAGGCTATAGTGTGTTGTTCCCATCCAGTGGACTGATGTCTCAGGGCTGGCCTGATCTGAGGTACCAGAGGCTGGGGTGGCGGCCGGCCCTTGGAGTGATCCAGGTTCAGGGTTAAGCTTTTCCTCCCCTCAGGGCAAGCCCCGGCCTCAGGTGACCTGGACCAAAGAGGGGCAGCCCCTGGCAGGCGAGGAGGTGAGCATCCGCAACAGCCCCACAGACACCATCCTGTTCATCCGGGCCGCTCGCCGCGTGCATTCAGGCACTTACCAGGTGACGGTGCGCATTGAGAACATGGAGGACAAGGCCACGCTGGTGCTGCAGGTTGTTGGTGCGTGGCCAAGGCCTCCTTGAGCCCCCTTGTTTCCCTTCCCTGGGCTGGGCTGCTGGGGCCAAGGGTGGGGCCAGGGGACCCAACCCACAGCTCACATTTTCCAGTCCACTGCCCCAGCAGGCCTGGCCCTGGTTGGCAGGGGTGGGGTGGTCCCTGGAGCCAGTGACCCCCTGCTCACTGTCAGGAGGCGTGGTGACCCAACTGGGTCTGTCTCCCGCAGACAAGCCAAGTCCTCCCCAGGATCTCCGGGTGACTGACGCCTGGGGTCTTAATGTGGCTCTGGAGTGGAAGCCACCCCAGGATGTCGGCAACACGGAGCTCTGGGGGTACACAGTGCAGAAAGCCGACAAGAAGACCATGGTGAGCCCAGGGTCTGGGGTCCCCACGTGCACCCTGCCTAGGCCCGGCAGACCCAGGCCGCAGCTACCCTTCACTGTCCTCACCGTGGACCCCTCACCCTCCCTCTGCTGATCTGAATCCCTCCATAGTAGGGGTGGAGATGCCAAGGGCACCCAGGAGAGGCTCTCGGCATCAGGAAGGGCCTGGCCCAGAGATGCCTCCCCTCCCTCCCTGCCCCCAGGAGTGGTTCACCGTCTTGGAGCATTACCGCCGCACCCACTGCGTGGTGCCAGAGCTCATCATTGGCAATGGCTACTACTTCCGCGTCTTCAGCCAGAATATGGTTGGCTTTAGTGACAGAGCGGCCACCACCAAGGAGCCCGTCTTTATCCCCAGACCAGGTGCTGTACCCTCATTCTTCCAACCAGGGGCTGGGGCAGGGAGGCTGTGGGAACAGGGAGAGGGGCCTAGCTTTGTGTGGCCCTCTCGGTACCAAGTCCTGTCACCAACAGGCATCACCTATGAGCCACCCAACTATAAGGCCCTGGACTTCTCCGAGGCCCCAAGCTTCACCCAGCCCCTGGTGAACCGCTCGGTCATCGCGGGCTACACTGCTATGCTCTGCTGTGCTGTCCGGGGTAGCCCCAAGGTAGGGAACTTTAGGCGCTGGTCCAGGCCGACCAGGAGGCAGGGCTCACAGGCCCCGACGTTGAGCAGTCCTCTCCCCAGCCTGTCTCCCCTGCTTTCTCTCCACCTTGGGGTGTATGGACCGGGTTCCTCCCCCTGGGCAGGGCCATGGTACTCACTCTTGGTTCCATGTTTGTTTCCAGCCTTGGGCATAGTCAGGGACTCTCGTGGGACCCCCCGAGTAGAAACACAGATGTGTCTCCCTGGGTCCCTGCCAGGTCCCCTCTCAGCCTGGATGGCTTCCCTCCCTCTCTTTACCTTATTTATAGCCCAAGATTTCCTGGTTCAAGAATGGCCTGGACCTGGGAGAAGACGCCCGCTTCCGCATGTTCAGCAAGCAGGGAGTGTTGACTCTGGAGATTAGAAAGCCCTGCCCCTTTGACGGGGGCATCTATGTCTGCAGGGCCACCAACTTACAGGGCGAGGCACGGTGTGAGTGCCGCCTGGAGGTGCGAGGTGAGGAGCCCTCGGGGCCAGGGCCTGGGAGATGGGAAGAGCGGGCGGCAGTGGGGACCCTGGGCTTTGCTCCGTTGTCCTCGGCCAAGCACCTGCCCTGGCTGCAAGGGCACGGACCCTGCCACGCCCAGATGGGCAATAGCTTCCAGAAGGCTGGGAGGACACAGTGACATGGCCTCCTCTTCTGCAGTGCCTCAGTGACCAGGCTGGCTCCTGGGGATGGCCAGGTATGTACCACCCGACACCCAGGGCACAAGTCAGTGGCTGAGCCCTGGCCCTTGGTCCTGGGGCAGCCATCAGTAAATGGGAGGCTGTAGGGGCCCTCCATTCACTCGTAAGATAACCTGTGTTGCAGGTACAACCGGATGCCAGCCCCGTGCCAGGAGCCTGGAGGGAAGTTGGGGAAACCCCTCCCTACTGTTGGATGTATGTGTGACAAGTGTGTCTCCTGTGCTGCGATGGGGGATCAGCAGGGCAGTTGTCGGGCAGTCCTGAGTGGGTGTTGCACAGACTGGTCCACAGGGCTCCTGAAGGAAGCCCCTGGATCTTTGGGGTAAAAGGAGGGTGGCCTCAAGAAACAATGTCTGGGGACAGGCCTTTCTGGCCTGCTATGTCTTCCCAATGTTTATTGGGCAATAAAAGATAAGTGCAGTCACAGAGAACTCACTCTTCTCAATTTCGGTGTGCCTGTTTTCATAAAAATGCACAGGGGATGGCCAGGTGCGGTGGCTCATGCCTGTTATCCCAGCACTTTGGGAGGCGGAGGCGGGCCGATCACCTGAGGTCAGGAGATCAAGACCAGCCTGGCCAACATGGTGAAACCCCGTCTCTACTAAAAATACAAAAATTAGCTGGGCAAGGTGGCGCGCCTATAATCCCAGCTATTTGGAAGGCTGAGGCAGGAGAATCACCTGAACCCAGGAGGTAGAGTTTGAGGTGGGCCGAGATCATGCCACTGCACTTCAGCCTGGGTGATGGAGTGAGACCATGTCTCAAAAAAAAAAAAAGAAAAAAGCCCAGGGACATTGATCTTCACAGCAAAGTGAGAGCAGATACTGTTTTTTGTTTTGTTTTCCCGATGATTAAACCAGGCCCAGAGTGGTTTCTGAGTTCATAAAACATTAACGCTAGAAGGAACCTGTCTTCCCCTACAGCATTCACTCTTCAGATGATGAAACTGAGCTGTGACAGCGGAAAGTGCATTTAACTGGTCCCAGGTGCTCAGCGTGATCCTTGTGCCAGGGGCCGTGGTCACAACCGCCTGGAGGTGGG